(19) United States
(12) Patent Application Publication
(10) Pub. No.: US 2015/0158911 A1
(43) Pub. Date: Jun. 11, 2015

(54) PIGMENT STRUCTURES, PIGMENT GRANULES, PIGMENT PROTEINS, AND USES THEREOF

(71) Applicants: Kevin Kit Parker, Cambridge, MA (US); Leila F. Deravi, Cambridge, MA (US); Evelyn Hu, Cambridge, MA (US); Andrew Magyar, Acton, MA (US)

(72) Inventors: Kevin Kit Parker, Cambridge, MA (US); Leila F. Deravi, Cambridge, MA (US); Evelyn Hu, Cambridge, MA (US); Andrew Magyar, Acton, MA (US)

(73) Assignee: PRESIDENT AND FELLOWS OF HARVARD COLLEGE, Cambridge, MA (US)

(21) Appl. No.: 14/118,716

(22) PCT Filed: May 18, 2012
(86) PCT No.: PCT/US12/38631

§ 371 Date: Feb. 23, 2015

Related U.S. Application Data
(60) Provisional application No. 61/488,370, filed on May 20, 2011.
(60) Provisional application No. 61/526,351, filed on Aug. 23, 2011.

(51) Int. Cl. C07K 7/08; A23L 1/275; A61K 8/64; A61Q 19/00; C07K 7/06; A61K 47/42

(52) U.S. Cl. CPC C07K 7/08; C07K 7/06; A61K 47/42; A61K 8/64; A61Q 19/00; A23L 1/2756; A61K 2800/434; A23V 2002/00

(57) ABSTRACT

The present invention is based, at least in part, on the isolation of intact pigment granules from the brown chromatophores in the skin of the cuttlefish, Sepia officinalis and the characterization of the optical properties of the isolated pigment granules. In particular, it has been discovered that the isolated pigment granules not only fluoresce in the far red wavelength of light when excited with blue/green light, but they also absorb and transmit or scatter light in the visible light range and are stable and optically active under ambient conditions.

### RELATED APPLICATIONS

This application claims priority to U.S. Provisional Application Ser. No. 61/488,370, filed on May 20, 2011 and U.S. 61/526,351, filed on Aug. 23, 2011, the entire contents of each of which are incorporated herein in their entirety by reference.

### GOVERNMENT SUPPORT

This invention was made with government support under National Science Foundation Grant No. PHY-0646094 and Department of Defense DARPA Grant No. W911NF-10-1-0113. The government has certain rights in the invention.

### BACKGROUND OF THE INVENTION

Efficient absorbance of light, particularly in reflection is critical for a number of technological applications. For example, light absorbance and reflection are critical for high performance photonic devices which include components, such as selective filters, polarizers, and low-threshold optical sources. Such components are currently formed from inorganic dielectric materials, such as semiconductors or metal oxides, which are costly and have minimal flexibility (R. M. Kramer, et al. (2007) Nature Materials 6:533; A. R. Tao, et al. (2010) Biomaterials 31:793; J. J. Walish, et al. (2009) Advanced Materials 21:3078).

Alternative materials which include colloidal photonic crystals have tremendously progressed leading to the fabrication of tunable optical devices that are broadly tunable over the optical spectrum and exhibit good reflectivity (˜40%). These devices, however, have technical shortcomings, specifically significant variation in their optical properties with thickness and large angular variation in coloration. Moreover, broad tunability of the optical response remains a challenge.

Accordingly, there is a need in the art for improved materials with desirable optical properties for use in photonic devices.

### SUMMARY OF THE INVENTION

The present invention is based, at least in part, on the isolation of intact pigment granules from the brown chromatophores in the skin of the cuttlefish, Sepia officinalis and the characterization of the optical properties of the isolated pigment granules. In particular, it has been discovered that the isolated pigment granules not only fluoresce in the far red wavelength of light when excited with blue/green light, but they also absorb and transmit or scatter light in the visible light range and are stable and optically active under ambient conditions.

It has also been discovered that the granules are composed largely of reflectin proteins but also contain, for example, lens proteins, myosin, actin, and intermediate filament proteins. Analysis of the granular chemical composition of the pigment granules isolated from S. officinalis brown chromatophores has demonstrated that the granular architecture of the pigment granule in concert with the high refractive index of the protein composition of the granule (i.e., reflectin and/or reflectin-like protein composition) results in the ability of the pigment granules to absorb and scatter light.

Finite difference time domain modeling has also demonstrated that the high refractive index of reflectin-based pigment granules enhances coloration and reflectance. The model shows that the high refractive index of the pigment granules increases reflectivity and color contrast in granules, mimicking reflectivity of pigment granules in the chromatophore organs. The isotropic arrangement and broad size distribution of pigment granules in chromatophores of S. officinallis, thus, leads to a large optical contrast through the combination of light scattering and absorbance. The nanoscale geometry of the pigment granules means that light experiences a longer path length as it travels through the chromatophore structure, thereby enhancing absorbance by the pigment contained within the granule. The absorbance by the pigment eliminates angular effects and minimizes spectral variation with thickness of the granular layer.

The present invention is also based, at least in part, on the discovery of novel peptides within the pigment granules isolated from the brown chromatophores in the skin of the cuttlefish, S. officinalis.

Accordingly, the present invention provides synthetic pigment structures that mimic the optical properties of the pigment granules isolated from the brown chromatophores in the skin of S. officinalis and uses thereof in, for example, photonic devices, adaptive textiles, and colorants. The present invention further provides proteins isolated from the isolated pigment granules.

In one aspect, the present invention provides pigment structures. The pigment structures include a reflectin protein and a light absorbing material.

The light absorbing material may be a dye, such as an inorganic dye or an organic dye and/or a fluorophore, such as an inorganic fluorophore or an organic fluorophore.

The reflectin protein may be selected from the group consisting of Euprymna scolopes reflectin 1a, Euprymna scolopes reflectin 1b, Euprymna scolopes reflectin 2a, Euprymna scolopes reflectin 2b, Euprymna scolopes reflectin 2c, Euprymna scolopes reflectin 3a, Doryteuthis pealeii reflectin-like A1, Doryteuthis pealeii reflectin-like A2, and Doryteuthis pealeii reflectin-like B1.

In another aspect, the present invention provides methods for preparing a pigment structure. The methods include providing a reflectin protein and a light absorbing material, combining the reflectin protein and the light absorbing material under conditions such that a reflectin protein nanostructure, e.g., a nanosphere, comprising the light absorbing material forms, thereby preparing a pigment structure.

The light absorbing material may be a dye, such as an inorganic dye or an organic dye and/or a fluorophore, such as an inorganic fluorophore or an organic fluorophore.

The reflectin protein for use in the methods of the invention may be selected from the group consisting of Euprymna scolopes reflectin 1a, Euprymna scolopes reflectin 1b, Euprymna scolopes reflectin 2a, Euprymna scolopes reflectin 2b, Euprymna scolopes reflectin 2c, Euprymna scolopes reflectin 3a, Doryteuthis pealeii reflectin-like A1, Doryteuthis pealeii reflectin-like A2, and Doryteuthis pealeii reflectin-like B1.

In yet another aspect, the present invention provides isolated pigment granules, which are isolated from a brown chromatophore of the chromatophore skin layer of Sepia officianalis.

The isolated pigment granule may have a diameter of about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, or about 2000 nm.

In one embodiment, the isolated pigment granule fluoresces.

In one embodiment, the isolated pigment granule which is excited with light having a wavelength of about 400, 401, 402, 403, 404, 405, 406, 407, 408, 409, 410, 411, 412, 413, 414, 415, 416, 417, 418, 419, or about 420 nm has a maximum emission at about 620, 630, 640, 650, 660, 670, 680, 690, 700, 710, 720, 730, 740, or about 750 nm.

In another embodiment, the isolated pigment granule which is excited with light having a wavelength of about 400, 401, 402, 403, 404, 405, 406, 407, 408, 409, 410, 411, 412, 413, 414, 415, 416, 417, 418, 419, or about 420 nm has a maximum emission between about 620-750, 620-720, 650-750, 620-700, or about 650-700 nm. In one embodiment, the isolated pigment granule which is excited with light having a wavelength of about 522, 523, 524, 525, 526, 527, 528, 529, 530, 531, 532, 533, 534, 535, 536, 537, 538, 539, 540, 541, or about 542 nm has a maximum emission at about 620, 630, 640, 650, 660, 670, 680, 690, 700, 710, 720, 730, 740, or about 750 nm.

In another embodiment, the isolated pigment granule which is excited with light having a wavelength of about 522, 523, 524, 525, 526, 527, 528, 529, 530, 531, 532, 533, 534, 535, 536, 537, 538, 539, 540, 541, or about 542 nm has a maximum emission between about 620-750, 620-720, 650-750, 620-700, or about 650-700 nm.

In one aspect, the present invention provides an isolated Sepia officinalis pigment protein, comprising the amino acid sequence YQDMMNMDFHGR (SEQ ID NO:1).

In another aspect, the present invention provides an isolated Sepia officinalis pigment protein, comprising the amino acid sequence YDNYGHDQYHGR (SEQ ID NO:2).

In yet another aspect, the present invention provides an isolated Sepia officinalis pigment protein, comprising the amino acid sequence LMYNNMYR (SEQ ID NO:3).

The isolated proteins of the invention may have a molecular weight less than about 10 kD, less than about 5 kD, or less than about 3 kD.

The present invention also provides isotropic thin films, nanofibers, nanofabrics, sensor, colorants, therapeutics, cosmetics, food products, and/or devices for diagnostic and/or therapeutic purposes which include the pigment structures and/or isolated pigment granules and/or pigment proteins of the invention.

### BRIEF DESCRIPTION OF THE DRAWINGS

FIG. 1 is a schematic of cephalopod chromatophores. A) Chromatophores are present in the cephalopod dermis and have yellow, red and brown pigment granules. B) Chromatophores are anchored radially by muscle fibers and are capable of ultrafast camouflage by contracting or expanding their chromatophores. C) Chromatophores are composed of nanospherical pigment granules tethered together by filaments or fibers. The exact composition of the pigment granules is unknown, but as described herein they are composed of a protein shell and an inert pigment core.

FIG. 2 are scanning electron micrographs of brown chromatophores (i) and pigment granules within the chromatophores (ii).

FIG. 3 is a schematic of the reflectivity of chromatophores. A) Light absorbed and reflected from the chromatophores is scattered in different ways depending on which color is expanded; yellow (i), red (ii), brown (iii). B) Percent reflectivity is red shifted depending on which color is absorbing and reflecting light. These data indicate that chromatophores behave as optical reflectors.

FIG. 4 is a schematic of pigment granule and pigment protein isolation from chromatophores. A) Whole cuttlefish Sepia officinalis are sacrificed and their dorsal mantle excised. The chromatophore layer is removed, digested, and purified using a percol gradient, which separates cellular components based on density. Pigment pellets contain pigment granules, which are imaged using scanning electron micrscopy to verify their purity. B) In isolating the pigment granules, chromatophores are lysed via ultrasonication, and pigment pellets are separated from the pigment supernatant. Both homogenates are loaded onto SDS-PAGE to purify proteins associated with the eluents. C) A portion of a gel lane for the pigment granule homogenate was excised and analyzed for protein content. D) An SDS-PAGE gel showing the fractions of a single lane purified for MS/MS analysis.

FIG. 5 are scanning electron micrographs at various magnifications of pigment granules isolated from the chromatophore which were immobilized onto a PDMS coated glass coverslip. The graph depicts the average diameter of the pigment granules.

FIG. 6A schematically depicts the fabrication of alginate gels comprising pigment granules. FIG. 6B depicts the luminescence of the alginate gels comprising the isolated pigment granules.

FIG. 7 is a Micro-Photoluminescence (MicroPL) graph demonstrating that the pigment granules themselves luminesce.

FIG. 8 depicts an exemplary device employing rotational motion for fabrication of nanofibers comprising isolated pigment granules and a micrograph of an exemplary fiber fabricated with an isolated pigment granule.

FIG. 9 depicts the photoluminescence associate with immobilized pigment granules. A) Excitation and emission sweeps collected from single pigment granules immobilized as a 2D film. B) Micro-Photoluminescence spectrograph of n=4 granules on 2 different substrates indicate variable emission profiles with peak centered at 650 nm.

FIG. 10 summarizes the luminescence of the isolated pigment granules immobilized different materials. A) Pigment granules immobilized as 2D films on PDMS substrates. (i) Granules are isotropic on substrates at the nanoscale as indicated through the Scanning Electron Micrographs (SEMs). (ii) Micro-Photoluminescence spectrograph of n=4 granules on 2 different substrates indicate variable emission profiles with peak centered at 650 nm B) Pigment granules processed as textiles embedded within a poly-Lactic Acid carrier system. Nanofibers were synthesized using rotary jet spinning (RJS) at 27,000 RPM. (i) Pseudo-colored granules processed as fibers. (ii) Micro-Photoluminescence spectrograph of n=4 granules on 2 different substrates indicate variable emission profiles with peak centered at 700 nm when compared to control (fiber with no pigment, green and purple lines). C) Pigment granules immobilized as 3D hydrogels in alginate. (i) Granules are isotropic within gels and demonstrate a structural iridescence. (ii) Micro-Photoluminescence spectrograph of n=4 granules on 2 different substrates indicate variable emission profiles with peak centered at 720 nm when compared to control (alginate not pigments, purple).

FIG. 11 depicts the effect of increasing concentrations of NaOH on the structure and function of the isolated pigment granules. Increasing NaOH concentration within a solution of granules A) (i) alters visible color of granules from black (left) to light red (right) and (ii) decreases absorbance. B) SEM of granules in increasing concentrations of NaOH indicates loss in secondary structure. Granules denatured in 0.2M NaOH appear mostly disrupted when compared to granular architecture at 0, 0.1, 0.5, and 0.8M concentrations. C) Gel electrophoresis of denatured granules indicates loss of protein content. D) Photoluminescence of granules denatured at 0.1M NaOH has highest luminescence intensity, and granules denatured at 0.2M NaOH have lowest. Emission maximum is gradually blue-shifted in the presence of increasing denaturant.

FIG. 12 depicts which proteins in the isolated pigment granules are affected by NaOH denaturation and are responsible for luminescence of the pigment granules.

FIG. 13 depicts the results of FDTD modeling of a chromatophore.

FIG. 14 schematically depicts the architecture of an optical device using pigment granules for colorations. Actuatable micron-sized membranes loaded with pigment granules with different absorption/scattering profiles would be stacked to create a tunable structure that can replicate the complete visible spectrum. The bottom layer is a perfect scatterer providing a diffuse white background. Above this is a “long pass” layer that absorbs the entire visible spectrum. Next is a “long pass” layer providing red coloration, and a “short pass” layer providing violet coloration. Above these are three reflective band layers, providing blue, green and yellow coloration. One of the possible spectral profiles for these layers is shown (right).

FIG. 15 schematically depicts a design for synthetic chromatophore. A) Principle mode of operation of the dielectric elastomer based chromatophore would be a voltage field. B) Upon application to the voltage field, the synthetic chromatophore will compress in the transverse directions and expand to induce a color change. C) Multiple synthetic chromatophores can be arranged in series to induce large scale color change.

FIG. 16 schematically depicts the preparation of synthetic pigment structures. A) Purified recombinant reflectin protein can self-assemble to form nanospheres in ionic solutions. This aggregation likely occurs due to electrostatic interactions of reflectin within the ionic solution 11-12. B) To synthesize a photonic nanostructure mimicking the chromatophore pigment granules, a negatively charge, hydrophobic fluorophore or dye is incorporated with the positively charged reflectin proteins. Because reflectin carries a positive charge under neutral pH, it spontaneously forms hybrid nanostructures with the negatively charged fluorophore to create a pigment granule mimetic.

### DETAILED DESCRIPTION OF THE INVENTION

The present invention is based, at least in part, on the isolation of intact pigment granules from the brown chromatophores in the skin of the cuttlefish, Sepia officinalis and the characterization of the optical properties of the isolated pigment granules. In particular, it has been discovered that the isolated pigment granules not only fluoresce in the far red wavelength of light when excited with blue/green light, but they also absorb and transmit or scatter light in the visible light range and are stable and optically active under ambient conditions.

In addition, the isotropic arrangement and broad size distribution of pigment granules in the chromatophores of S. officinalis provides a large optical contrast through a combination of light scattering and light absorbance.

It has also been discovered that the granules are composed largely of reflectin proteins but also contain, for example, lens proteins, myosin, actin, and intermediate filament proteins. Analysis of the granular chemical the granular chemical composition of the pigment granules isolated from S. officinalis brown chromatophores demonstrated that the granular architecture of the pigment granule in concert with the high refractive index of the protein composition of the granule (i.e., reflectin and/or reflectin-like protein composition) results in the ability of the pigment granules to absorb and scatter light.

Finite difference time domain modeling also demonstrated that the high refractive index of reflectin-based pigment granules enhances coloration and reflectance. The model shows that the high refractive index of the pigment granules increases reflectivity and color contrast in granules, mimicking reflectivity of pigment granules in the chromatophore organs.

The present invention is also based, at least in part, on the discovery of novel proteins within the pigment granules isolated from the brown chromatophores in the skin of the cuttlefish, S. officinalis.

Accordingly, the present invention provides synthetic pigment structures that mimic the optical properties of the pigment granules isolated from the brown chromatophores in the skin of S. officinalis and uses thereof in, for example, thin films, nanofibers, nanofabrics, sensor, colorants, therapeutics, cosmetics, food products, and/or devices for diagnostic and/or therapeutic purposes. The present invention further provides isolated pigment granules from the brown chromatophores of the skin of S. officinalis and uses thereof, as well as proteins isolated from the isolated pigment granules.

### I. Synthetic Pigment Structures

As described in the Examples section, it has been discovered that the ability of the isolated pigment granules from S. officinalis brown chromatophores to absorb and scatter light is due to the granular architecture of the pigment granule in concert with the high refractive index of the protein composition of the granule (i.e., reflectin and/or reflectin-like protein composition).

In addition, the isotropic arrangement and broad size distribution of pigment granules in the chromatophores of S. officinalis provides a large optical contrast through a combination of light scattering and light absorbance. Therefore, light experiences a longer path length as it travels through the chromatophore structure and the pigment granules, thereby enhancing absorbance by the pigment proteins contained within the pigment granule. In addition, the absorbance of light by the pigment protein eliminates the angular effects and minimizes spectral variation with thickness of the granular layer.

Accordingly, in one aspect, the present invention provides synthetic pigment structures that mimic the granular structure and the optical characteristics of the pigment granules in the brown (or sepia) chromatophores of S. officinalis skin.

In one embodiment, the pigment structure of the present invention comprises a reflectin protein (and/or a reflectin-like protein) and a light absorbing material, such as a dye, e.g., an organic or inorganic dye, or a fluorophore, e.g., an organic or inorganic fluorophore. In one embodiment, the reflectin protein is hydrophillic and positively charged. In one embodiment, the dye and/or fluorophore is hydrophobic and negatively charged.

The terms reflectin protein, as used herein, refers to both reflectin proteins and reflectin-like proteins unless otherwise specified (see below).

The pigment structures of the present invention are prepared by combining a reflectin and/or reflectin-like protein and the light absorbing material under conditions such that a reflectin protein nanostructure, e.g., nanosphere, forms. Accordingly, the present invention provides methods for preparing a pigment structure, comprising providing a reflectin protein and a light absorbing material, combining the reflectin protein and the light absorbing material under conditions such that a reflectin protein nanostructure, e.g., nanosphere, comprising the light absorbing material forms, thereby preparing a pigment structure.

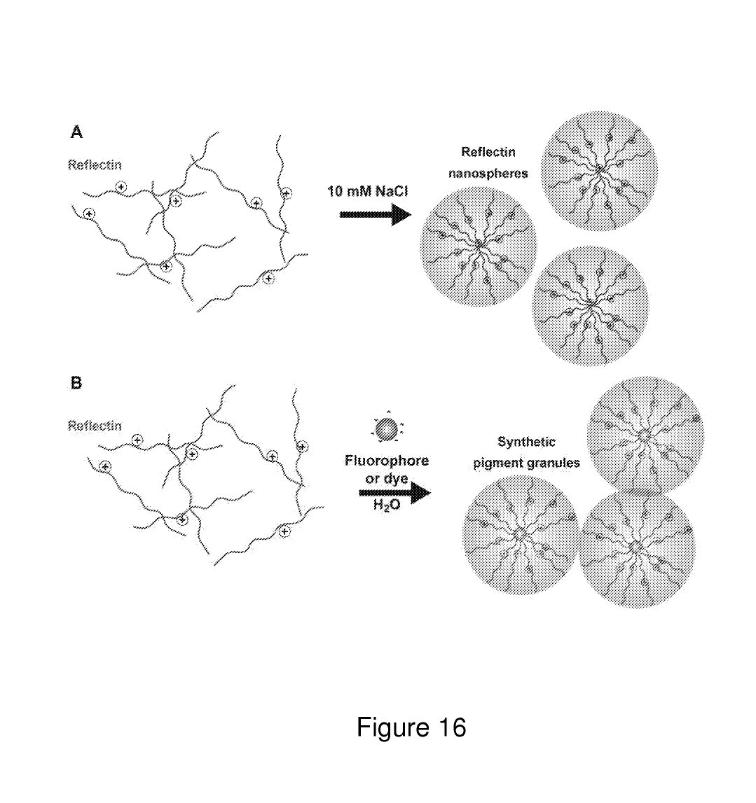

For example, a solution of the reflectin protein and a solution of the light absorbing material are combined until a reflectin protein nanostructure, e.g., nanosphere, forms. Alternatively, a light absorbing material, e.g., a dye and/or fluorophore, may be added as dry material to a solution of the reflectin protein. The solutions of one or both of the reflectin protein and the light absorbing material may be aqueous solutions having a neutral pH. The reflectin proteins (e.g., a hydrophillic protein) will spontaneously form nanostructures, e.g., nanospheres, which incorporate the light absorbing material (e.g., a hydrophobic dye and/or fluorophore) (see FIG. 16). Due to the high refractive index of the proteins and the confinement of the light absorbing material to the inner portion of the pigment structure, the optical efficiency of the light absorbing material will be enhanced.

For example, an aqueous solution of reflectin and/or a light absorbing material may be buffered and comprise a salt, such as, but not limited to sodium chloride, sodium citrate, sodium phosphate, at a concentration of about 0, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10 mM. Values intermediate to the above recited values are also contemplated to be part of the invention. The charge of the fluorophore and/or dye will dictate the concentration and type of buffer required to form dye/fluorophore containing reflectin nanospheres. For example, if a fluorophore or dye is negatively charged, the solution will not contain any buffer (e.g., ions) so that the positively charged reflectin protein can assemble into nanospheres with the negatively charged fluorophore via electrostatic interactions. If a fluorophore or dye is positively charged, the solution will contain a buffer having negative ions so that the positively charged reflectin protein can assemble into nanospheres with the positively charged fluorophore via electrostatic interactions.

The concentration of reflectin and/or reflectin-like protein for use in the methods of the invention may be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10.5, 11, 11.5, 12, 12.5, 13, 13.5, 14, 14.5, 15, 15.5, 16, 16.5, 17, 17.5, 18, 18.5, 19, 19.5, 20, 21, 22, 23, 24, 25, 26, 27, 28, 29, 30, 31, 32, 33, 34, 34, 35, 36, 37, 38, 39, 40, 41, 42, 43, 44, 45, 46, 47, 48, 49, or about 50 mg/mL. Values intermediate to the recited values are also intended to be part of this invention.

The diameter of a reflectin nanosphere is inversely proportional to the concentration of the refelectin protein used to prepare the nanospheres. For example, a solution having 10 mg/ml reflectin protein will spontaneously form nanspheres having a diameter of about 7 nm and a solution having 50 mg/ml reflectin protein will spontaneously form nanspheres having a diameter of about 5 nm. As the diameter of the light absorbing materials suitable for use in the present invention, have a diameter of about 10-100 nm, the pigment structures of the invention comprising a reflectin protein and a light absorbing material may range from about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, or about 2000 nm. Values intermediate to the recited values are also intended to be part of this invention.

The pigment structures of the present invention may be combined with any suitable additional material to create photonic devices, adaptive textiles, and colorants. The uses of the pigment structures are described in detail below.

Reflectin proteins suitable for use in the pigment structures and compositions comprising such pigment structures are known in the art and include reflectin proteins derived from Euprymna scolopes, or variants thereof, and reflectin-like proteins derived from Doryteuthis (formerly Loligo) pealeii, variants thereof, or biologically active fragments thereof. The reflectin and reflectin-like polypeptides isolated from S. officinalis described herein are also suitable for use in the pigment structures. Recombinantly or synthetically produced reflectin proteins or fragments or variants thereof are suitable for use in the compositions and methods of the invention.

Numerous isoforms of reflectin have been identified in Euprymna scolopes and include reflectin 1a, reflectin 1b, reflectin 2a, reflectin 2b, reflectin 2c, and reflectin 3a. The nucleotide and amino acid sequences of these reflectin proteins are described in U.S. Pat. No. 7,314,735, the entire contents of which are incorporated herein by reference.

Numerous isoforms of reflectin-like proteins have been identified in Doryteuthis pealeii and include reflectin-like A1, reflectin-like A2, and reflectin-like B1. The nucleotide and amino acid sequences of these reflectin proteins are known in the art and may be found in, for example, GenBank Accession Nos.: GI:269996957, GI:269996959, and GI:269996961, respectively.

Native reflectin and/or reflectin-like proteins can be isolated from cells or tissue sources by an appropriate purification scheme using standard protein purification techniques. Reflectin and/or reflectin-like proteins may also be produced by recombinant DNA techniques. Alternative to recombinant expression, reflectin and/or reflectin-like proteins or polypeptides can be synthesized chemically using standard peptide synthesis techniques.

An “isolated” or “purified” protein or biologically active portion thereof is substantially free of cellular material or other contaminating proteins from the cell or tissue source from which the pigment protein is derived, or substantially free from chemical precursors or other chemicals when chemically synthesized.

Biologically active portions of a reflectin and/or reflectin-like protein include peptides comprising amino acid sequences sufficiently identical to or derived from the amino acid sequence of the reflectin and reflectin-like proteins which include less amino acids than the full length reflectin and reflectin-like proteins, and exhibit at least one activity of a reflectin or reflectin-like proteins, e.g., spontaneous formation of protein nanospheres and/or reflection of visible light.

Other useful reflectin and reflectin-like proteins are substantially identical to the reflectin and reflectin-like proteins described above and retain the functional activity of the protein yet differ in amino acid sequence due to natural allelic variation or mutagenesis.

A useful reflectin and/or reflectin-like protein is a protein which includes an amino acid sequence at least about 45, 55, 65, 75, 76, 77, 78, 79, 80, 81, 82, 83, 84, 85, 86, 87, 88, 89, 90, 91, 92, 93, 94, 95, 96, 97, 98, or 99% identical to the amino acid sequence of the reflectin and reflectin-like proteins described above, and retains the functional activity of the reflectin and/or reflectin-like proteins.

Methods to determine the percent identity of two amino acid sequences or of two nucleic acids are described below.

A light absorbing material (and/or reflectin-like protein) may be identified using methods routine to one of ordinary skill in the art. Such methods include, for example, use of a refractometer, e.g., a traditional handheld refractometer, adigital handheld refractometer, a laboratory or Abbe refractometer, or an inline process refractometer. The index of refraction may calculated from Snell's law and/or from the composition of the material using the Gladstone-Dale relation.

Suitable light absorbing dyes to incorporate into the pigment structures for use in the present invention are known and include, for example, inorganic dyes, such as iron oxide, cobalt(II) oxide, titanium yellow, ultramarine, and Prussian blue. Organic dyes suitable for use in the present invention include, for example, ommochromes, melanin, corroles, methylene blue (and derivatives thereof), carbon black, alizarin (and derivatives thereof), carmine (and derivatives thereof), and indigo.

Suitable fluorophores to incorporate into the pigment structures for use in the present invention are known in the art and include, for example, GFP proteins (and derivatives thereof), quantum dots, fluorescein (and derivatives thereof), coumarin (and derivatives thereof), rhodamine, and porphyrin.

Depending on the desired application of the synthetic pigment granules, one of ordinary skill in the art may readily choose an appropriate dye and/or fluorophore based on the known optical properties of the dye and/or fluorophore. Furthermore, by choosing a dye and/or fluorophore with a particular spectral signature the absorbance and emission properties of the pigment structure can be controlled enabling the synthesis of pigment structures having narrow spectral resonances for optical filters or broad spectral resonances for displays and solar cells.

For example, iron oxide absorbs light with wavelengths longer than about 650 nm, cobalt(II) oxide absorbs light with wavelengths longer than about 515 nm, titanium yellow absorbs light with wavelengths longer than about 525 nm, ultramarine has a maximum light absorbance at about 450 nm, Prussian blue has a maximum light absorbance at about 680 nm, ommochromes have a maximum light absorbance at about 520 nm, melanin has a maximum light absorbance at about 335 nm, corroles have a maximum light absorbance at about 498 nm, methylene blue (and derivatives thereof) has a maximum light absorbance at about 670 nm, carbon black is a broadband light absorber, alizarin (and derivatives thereof) has a maximum light absorbance at about 250 and 450 nm, carmine (and derivatives thereof) has a maximum light absorbance at about 600 nm, and indigo has a maximum light absorbance at about 275 and 625 nm. GFP proteins (and derivatives thereof) have an excitation wavelength of about 395 and 475 nm and an emission wavelength of about 509 nm, quantum dots have an excitation wavelength of about 295 nm-850 nm, an emission wavelength of about 300-900 nm, and a maximum light absorbance of about 500-600 nm, fluorescein (and derivatives thereof) have an excitation wavelength of about 494 nm and an emission wavelength of about 521 nm, coumarin (and derivatives thereof) have an excitation wavelength of about 450, an emission wavelength of about 500 nm, and a maximum light absorbance of about 450 nm, rhodamine has an excitation wavelength of about 522 nm, an emission wavelength of about 550 nm, and a maximum light absorbance of about 500 nm, and porphyrin has an excitation wavelength of about 340-550 nm, an emission wavelength of about 400-750 nm, and a maximum light absorbance of about 400 nm, all dependent on the metal center and/or side groups.

For example, for narrow band applications, ither quantum dots or rhodamine derivatives are useful. For broad band applications, pyrroles such as methylene blue are useful light absorbing materials.

The optical properties of any dye or fluorophore may be determined using methods routine to one of ordinary skill in the art and include, for example, use of any standard spectroscopic technique as described herein and include use of instruments such as photoluminescence or luminescence spectrometers, or fluorescence spectrophotometers.

II. Isolated Sepia officinalis Pigment Granules

As described below, intact pigment granules from the brown chromatophores of the chromatophore skin layer of S. officianalis have been isolated and characterized. The isolation methods include dissection of the epidermal and iridiphore layers from dorsal mantle skin of S. officianalis, ultrasonication of the remaining skin tissue comprising the chromatophore layer (e.g., red, yellow and brown chromatophores), and purification of the skin homogenate through a sucrose gradient. The brown chromatophores, which are the densest material due to the tight packing of pigment granules, were pelleted and separated from the red and yellow chromatophores which remain in the supernatant. Once the supernatant was removed, the pigment pellet was collected, sonicated and further purified by centrifugation. The resulting pigment pellet was re-suspended in water. The isolated brown chromatophores were lysed via ultrasonication, which is a process that is strong enough to rupture the chromatophore membrane but not strong enough to destroy the pigment granules.

Accordingly, the present invention provides methods for the isolation of brown chromatophores from the chromatophore skin layer of S. officianalis. The methods include providing a skin tissue sample, ultrasonication of the tissue sample such that a homogenized tissue sample is prepared, gradient centrifugation of the homogenized tissue sample, such that a pellet of brown chromatophores is prepared, thereby isolating brown chromatophores from the chromatophore skin layer of S. officianalis. The methods may include additional steps, such as sonication and washing.

The pigment granules isolated according to the methods described herein have been physically characterized and it has been demonstrated that the isolated pigment granules have an average diameter of about 930 nm+/−250 nm. Analysis of the optical qualities of the isolated pigment granules indicate that these pigment granules are not only excitable, emitting photons around 390 nm to about 750 nm (i.e., fluoresce) but also absorb and scatter visible light. In particular, the isolated pigment granules luminesce in the far red with a maximum emission centered broadly at about 650 nm (e.g., between about 625 nm to about 750 nm) with a maximal excitation at about 410 nm and about 532 nm. Emission from pigment granules isolated form yellow and red chromnatophores can be distinguished from those isolated from the brown chromatophores as they have both peak a flouresence peak at about 620 nm.

Accordingly, in one aspect, the present invention provides an isolated pigment granule, wherein the pigment granule is isolated from a brown chromatophore of the chromatophore skin layer of Sepia officianalis. The isolated pigment granule may have a diameter of about 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, or about 1180 nm.

Methods to determine the diameter of an isolated pigment granule include standard microscopic methods, such as scanning electron microscopy and bright field microscopy.

In one embodiment, the isolated pigment granule fluoresces.

Methods to determine if an isolated pigment granule fluoresces are described herein and include standard use of MicroPhotoluminescence or photoluminescence.

In another embodiment, the luminescence from the isolated pigment granule can be excited with light having a wavelength of about 400, 401, 402, 403, 404, 405, 406, 407, 408, 409, 410, 411, 412, 413, 414, 415, 416, 417, 418, 419, or about 420 nm with a maximum emission at about 620, 630, 640, 650, 660, 670, 680, 690, 700, 710, 720, 730, 740, or about 750 nm, or between about 620-750, 620-720, 650-750, 620-700, or about 650-700 nm. In another embodiment, the luminescence from the isolated pigment granule can be excited with light having a wavelength of about 522, 523, 524, 525, 526, 527, 528, 529, 530, 531, 532, 533, 534, 535, 536, 537, 538, 539, 540, 541, or about 542 nm with a maximum emission at about 620, 630, 640, 650, 660, 670, 680, 690, 700, 710, 720, 730, 740, or about 750 nm, or between about 620-750, 620-720, 650-750, 620-700, or about 650-700 nm.

Methods to determine the excitation and emission wavelengths an isolated pigment granule fluoresces are also described herein and include standard use of microphotoluminescence excitation (PLE) spectroscopy.

### III. Pigment Proteins

In another aspect, the present invention provides proteins (or polypeptides) isolated from the pigment granules in the brown chromatophore of the chromatophore layer of the skin of S. officinalis, and biologically active fragments thereof. Proteins isolated from the pigment granules of the S. officinalis brown chromatophore were separated using a polyacrylamide gel having a gradient of increasing acrylamide concentration (i.e., Criterion Tris-HCl 4-15% Polyacrylamide Gel (Catalog #345-0028)), sections of the gel were isolated, trypsinized and the cleaved peptides were sequenced using mass spectroscopy.

The sequences were analyzed and the proteins identified from the cuttlefish tissue include those having significant amino acid identity (<50%) to the reflectin family of proteins (1a, 2a, 3), crystallin proteins, muscle proteins, such as actin and myosin, intermediate filament proteins, as well as three proteins corresponding to new members of the reflectin family of proteins (see Table 1).

TABLE 1

| SEQUENCE | AMINO ACID SIMILARITY TO: | SEQ ID NO: |
|---|---|---|
| YQDMMNMDFHGR | Reflectin-like protein B1 | 1 |
| | (Loligo pealei) | |
| YDNYGHDQYHGR | Reflectin-like protein Al | 2 |
| | (Loligo pealei) | |
| LMYNNMYR | Reflectin-like protein A2 | 3 |
| | (Loligo pealei) | |

Reflectin and reflectin-like protein family members typically have an amino acid composition enriched in aromatic and sulphur-containing amino acids. In some embodiments, reflectin and reflectin-like proteins have amino acid compositions in which a majority of the amino acid residues are rare amino acid residues (i.e., tyrosine, methionine, arginine, and tryptophan) and often lack any common amino acid residues (i.e., alanine, isoleucine, leucine and lysine). Reflectin proteins also contain repeating (e.g., about five) reflective repeat peptide motifs having an amino acid sequence of [a(X)₄₋₅MD(X)₅MD(X)₃₋₄] (SEQ ID NO:4), wherein a is the amino acid sequence MD, FD or null, and X is any amino acid. Reflectin-like proteins may also include methionine repeats, similar to those in methionine rich repeat proteins, or mrrp proetins.

The three new reflectin protein family members identified herein were all isolated from a portion of the 4-15% gradient gel estimated to contain proteins having a molecular weight of less than about 10 kD, less than about 9 kD, less than about 8 kD, less than about 7 kD, less than about 6 kD, less than about 5 kD, or about 2.5 kD to about 5 kD, about 3 kD to about 5 kD, about 3.5 kD to about 5 kD, about 3 kD to about 4.5 kD, or about 3 kD to about 4 kD.

Accordingly, in one aspect, the present invention provides an isolated Sepia officinalis pigment protein, comprising the amino acid sequence YQDMMNMDFHGR (SEQ ID NO:1). In another aspect, the present invention provides an isolated Sepia officinalis pigment protein, comprising the amino acid sequence YDNYGHDQYHGR (SEQ ID NO:2). In yet another aspect, the present invention provides an isolated Sepia officinalis pigment protein, comprising the amino acid sequence LMYNNMYR (SEQ ID NO:3).

In one embodiment, an isolated Sepia officinalis pigment protein, comprises 1, 2, 3, 4, 5, 6, or 7 reflective repeat peptide motifs having an amino acid sequence of [a(X)₄₋₅MD(X)₅MD(X)₃₋₄], wherein a is the amino acid sequence MD, FD or null, and X is any amino acid.

In one embodiment, the present invention provides an isolated Sepia officinalis pigment protein, comprising the amino acid sequence YQDMMNMDFHGR (SEQ ID NO:1) which is about 2.5 kD to about 5 kD, as determined by use of 4-15% gradient gel electrophoresis. In another embodiment, the present invention provides an isolated Sepia officinalis pigment protein, comprising the amino acid sequence YQDMMNMDFHGR (SEQ ID NO:1) which is about 2.5 kD to about 5 kD, as determined by use of 4-15% gradient gel electrophoresis and comprises 1, 2, 3, 4, 5, 6, or 7 reflective repeat peptide motifs having an amino acid sequence of [a(X)₄₋₅MD(X)₅MD(X)₃₋₄], wherein a is the amino acid sequence MD, FD or null, and X is any amino acid.

In another embodiment, the present invention provides an isolated Sepia officinalis pigment protein, comprising the amino acid sequence YDNYGHDQYHGR (SEQ ID NO:2) which is about 2.5 kD to about 5 kD, as determined by use of 4-15% gradient gel electrophoresis. In another embodiment, the present invention provides an isolated Sepia officinalis pigment protein, comprising the amino acid sequence YDNYGHDQYHGR (SEQ ID NO:2) which is about 2.5 kD to about 5 kD, as determined by use of 4-15% gradient gel electrophoresis and comprises 1, 2, 3, 4, 5, 6, or 7 reflective repeat peptide motifs having an amino acid sequence of [a(X)₄₋₅MD(X)₅MD(X)₃₋₄], wherein a is the amino acid sequence MD, FD or null, and X is any amino acid.

In yet another embodiment, the present invention provides an isolated Sepia officinalis pigment protein, comprising the amino acid sequence LMYNNMYR (SEQ ID NO:3) which is about 2.5 kD to about 5 kD, as determined by use of 4-15% gradient gel electrophoresis. In another embodiment, the present invention provides an isolated Sepia officinalis pigment protein, comprising the amino acid sequence LMYNNMYR (SEQ ID NO:3) which is about 2.5 kD to about 5 kD, as determined by use of 4-15% gradient gel electrophoresis and comprises 1, 2, 3, 4, 5, 6, or 7 reflective repeat peptide motifs having an amino acid sequence of [a(X)₄₋₅MD(X)₅MD(X)₃₋₄], wherein a is the amino acid sequence MD, FD or null, and X is any amino acid.

S. officinalis pigment proteins and polypeptides can be isolated from cells or tissue sources by an appropriate purification scheme using standard protein purification techniques as described herein. S. officinalis pigment polypeptides may also be produced by recombinant DNA techniques. Alternative to recombinant expression, a S. officinalis pigment protein or polypeptide can be synthesized chemically using standard peptide synthesis techniques.

An “isolated” or “purified” protein or biologically active portion thereof is substantially free of cellular material or other contaminating proteins from the cell or tissue source from which the pigment protein is derived, or substantially free from chemical precursors or other chemicals when chemically synthesized. The language “substantially free of cellular material” includes preparations of pigment protein in which the protein is separated from cellular components of the cells from which it is isolated or recombinantly produced. Thus, a pigment protein that is substantially free of cellular material includes preparations of pigment protein having less than about 30%, 20%, 10%, or 5% (by dry weight) of non-pigment protein. When the pigment protein or biologically active portion thereof is recombinantly produced, it is also preferably substantially free of culture medium, i.e., culture medium represents less than about 20%, 10%, or 5% of the volume of the protein preparation. When pigment protein is produced by chemical synthesis, it is preferably substantially free of chemical precursors or other chemicals, i.e., it is separated from chemical precursors or other chemicals which are involved in the synthesis of the protein. Accordingly such preparations of pigment protein have less than about 30%, 20%, 10%, 5% (by dry weight) of chemical precursors or non-pigment protein chemicals.

Biologically active portions of a pigment protein include peptides comprising amino acid sequences sufficiently identical to or derived from the amino acid sequence of the pigment protein (e.g., the amino acid sequence shown in SEQ ID NO:1, SEQ ID NO:2, or SEQ ID NO:3), which include less amino acids than the full length SEQ ID NO:1, SEQ ID NO:2, or SEQ ID NO:3 and retain the functional activity of the protein of SEQ ID NO:1, SEQ ID NO:2, or SEQ ID NO:3 (e.g., spontaneously form nanospheres in neutral pH solutions and/or reflect visible light).

Other useful pigment polypeptides are substantially identical to SEQ ID NO:1, SEQ ID NO:2, or SEQ ID NO:3 and retain the functional activity of the protein of SEQ ID NO:1, SEQ ID NO:2, or SEQ ID NO:3 yet differ in amino acid sequence due to natural allelic variation or mutagenesis.

A useful pigment protein is also a protein which includes an amino acid sequence at least about 45%, 55, 65, 75, 76, 77, 78, 79, 80, 81, 82, 83, 84, 85, 86, 87, 88, 89, 90, 91, 92, 93, 94, 95, 96, 97, 98, or 99% identical to the amino acid sequence of SEQ ID NO:1, SEQ ID NO:2, or SEQ ID NO:3, and retains the functional activity of the SEQ ID NO:1, SEQ ID NO:2, or SEQ ID NO:3.

Additionally, useful pigment proteins include full-length reflectin and/or reflectin-like proteins comprising the amino acid sequence of SEQ ID NO:1, SEQ ID NO:2, or SEQ ID NO:3, which retain the functional activity of the SEQ ID NO:1, SEQ ID NO:2, or SEQ ID NO:3.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions×100).

The determination of percent homology between two sequences can be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences similar or homologous to pigment protein and/or nucleic acid molecules encoding pigment proteins of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. When utilizing the ALIGN program for comparing nucleic acid sequences, a gap length penalty of 12, and a gap penalty of 4 can be used. Another preferred example of a mathematical algorithm utilized for the comparison of sequences is the Needleman and Wunsch (J. Mol. Biol. (48):444-453 (1970)) algorithm which has been incorporated into the GAP program in the GCG software package (available at gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at gcg.com), using a NWSgapdna.CMP matrix and a gap weight of 40, 50, 60, 70, or 80 and a length weight of 1, 2, 3, 4, 5, or 6.

The percent identity between two sequences can be determined using techniques similar to those described above, with or without allowing gaps. In calculating percent identity, typically exact matches are counted.

The invention also provides pigment chimeric or fusion proteins. As used herein, a “pigment chimeric polypeptide” or “pigment fusion protein” comprises a pigment protein operatively linked to a non-pigment polypeptide. A “pigment polypeptide” refers to a polypeptide having an amino acid sequence corresponding to all or a portion (preferably a biologically active portion) of a SEQ ID NO:1, SEQ ID NO:2, or SEQ ID NO:3 or an amino acid sequence comprising SEQ ID NO:1, SEQ ID NO:2, or SEQ ID NO:3, e.g., a full-length reflectin or reflectin-like protein, whereas a “non-pigment polypeptide” refers to a polypeptide having an amino acid sequence corresponding to a protein which is not substantially identical to the SEQ ID NO:1, SEQ ID NO:2, or SEQ ID NO:3, or an amino acid sequence comprising SEQ ID NO:1, SEQ ID NO:2, or SEQ ID NO:3, e.g., a full-length reflectin or reflectin-like protein, e.g., a protein which is different from the pigment proteins and which is derived from the same or a different organism. Within the fusion protein, the term “operatively linked” is intended to indicate that the pigment polypeptide and the non-pigment polypeptide are fused in-frame to each other. The heterologous polypeptide can be fused to the N-terminus or C-terminus of the pigment polypeptide.

A pigment protein chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (e.g., a GST polypeptide). A pigment prtein-encoding nucleic acid can be cloned into such an expression vector such that the fusion moiety is linked in-frame to the pigment protein.

Another aspect of the invention pertains to isolated nucleic acid molecules that encode S. officinalis pigment proteins or biologically active portions thereof, or a complement thereof, as well as nucleic acid molecules sufficient for use as hybridization probes to identify S. officinalis pigment protein-encoding nucleic acids and fragments for use as PCR primers for the amplification or mutation of S. officinalis pigment proteins nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one that is separated from other nucleic acid molecules, which are present in the natural source of the nucleic acid. Preferably, an “isolated” nucleic acid is free of sequences (preferably protein encoding sequences) that which naturally flank the nucleic acid (i.e., sequences located at the 5′ and 3′ ends of the nucleic acid) in the genomic DNA of the organism from which the nucleic acid is derived. For example, in various embodiments, the isolated S. officinalis pigment proteins nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule encoding the amino acid sequence of SEQ ID NO:1, SEQ ID NO:2, and/or SEQ ID NO:3, or an amino acid sequence comprising SEQ ID NO:1, SEQ ID NO:2, or SEQ ID NO:3, e.g., a full-length reflectin or reflectin-like protein, or a complement of any of these nucleotide sequences, can be isolated using standard molecular biology techniques and the sequence information provided herein. Using all or portion of the nucleic acid sequences of SEQ ID NO:1, SEQ ID NO:2, or SEQ ID NO:3 nucleic acid molecules can be isolated using standard hybridization and cloning techniques (e.g., as described in Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

A nucleic acid of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to S. officinalis pigment protein encoding nucleotide sequences can be prepared by standard synthetic techniques, e.g., using an automated DNA synthesizer.

In another embodiment, an isolated nucleic acid molecule of the invention comprises a nucleic acid molecule which is a complement of the nucleotide sequence encoding a pigment protein set forth in SEQ ID NO:1, SEQ ID NO:2, and/or SEQ ID NO:3, or a portion thereof, or a full-length reflectin or reflectin-like protein comprising SEQ ID NO:1, SEQ ID NO:2, and/or SEQ ID NO:3. A nucleic acid molecule which is complementary to a given nucleotide sequence is one which is sufficiently complementary to the given nucleotide sequence that it can hybridize to the given nucleotide sequence thereby forming a stable duplex.

Moreover, the nucleic acid molecule of the invention can comprise only a portion of a nucleic acid sequence encoding SEQ ID NO:1, SEQ ID NO:2, and/or SEQ ID NO:3, for example, a fragment which can be used as a probe or primer or a fragment encoding a biologically active portion of a pigment protein.

Probes based on the nucleotide sequences encoding the pigment proteins of the invention can be used to detect transcripts or genomic sequences encoding the same or similar proteins. The probe comprises a label group attached thereto, e.g., a radioisotope, a fluorescent compound, an enzyme, or an enzyme co-factor.

IV. Uses of Synthetic Pigment Structures and Isolated S. officinalis Pigment Granules

The synthetic pigment structures, isolated pigment granules, and isolated proteins (e.g., the proteins of SEQ ID NOs:1-3 or proteins comprising these sequences) described herein offer numerous advantages over current optical materials. For example, efficient absorbance of light, particularly in reflection, is critical for a number of technological applications. Researchers studying colloidal photonic crystals have made tremendous progress creating tunable optical devices that are broadly tunable over the optical spectrum and exhibit good reflectivity (˜40%). However, these devices have significant variation in the optical properties with thickness and large angular variation in coloration. In addition, quantum dots are toxic to cells.

In contrast, the synthetic pigment structures and isolated pigment granules described herein have robust properties, such as being optically active and stable at room temperature and in aqueous solutions, are energetically efficient and can be used for reflection and absorption, are non-toxic (bio-compatible). Thus, the synthetic pigment structures and isolated pigment granules described herein can be used in biosensors, biological imaging (similar to Green fluorescent protein), electronic displays, light emitting diodes, optical fibers and textiles (e.g., camouflage), photodetectors, paints, cosmetics, optics, nanophotonic computers, computational machines, and colorants for, e.g., paint and food products.

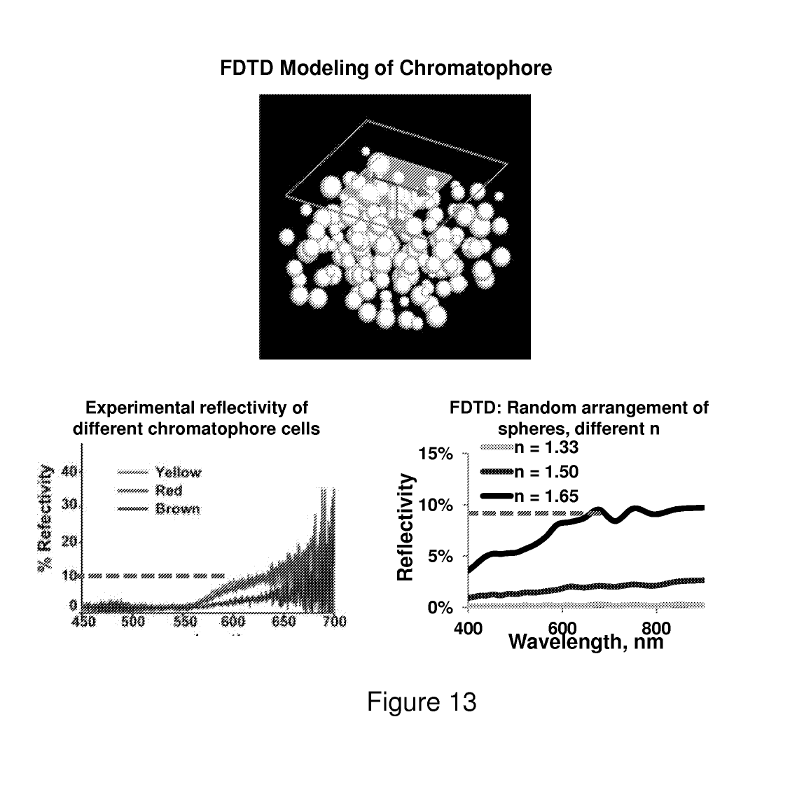

In particular, optical displays, such as those for e-readers, rapidly tunable optical filters for information processing, or even structures to enhance light absorption in solar cells can be fabricated using the synthetic pigment structures and isolated pigment granules described herein (see FIG. 14). For example, actuatable micron-sized membranes loaded with synthetic pigment structures and/or pigment granules having different absorption/scattering profiles may be stacked to create a tunable structure that can replicate the complete visible spectrum. As shown in FIG. 14, the bottom layer is a perfect scatterer providing a diffuse white background. Above this is a “long pass” layer that absorbs the entire visible spectrum. Next is a “long pass” layer providing red coloration, and a “short pass” layer providing violet coloration. Above these are three reflective band layers, providing blue, green and yellow coloration. By selectively expanding or contracting the individual layers the entire optical spectrum can be created with high optical contrast.

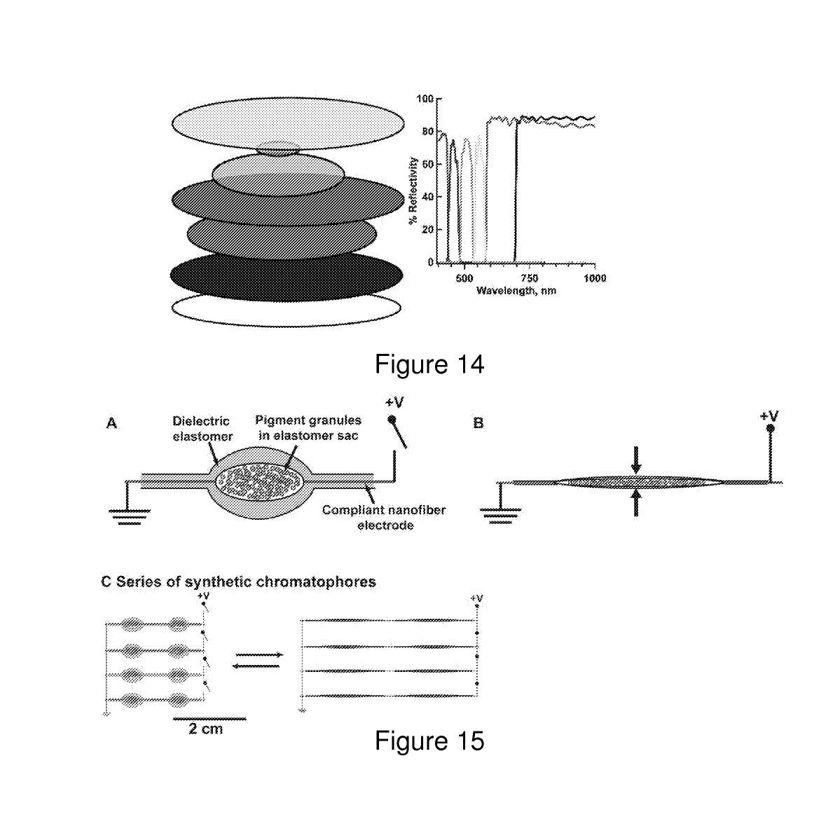

Synthetic chromatophores can also be engineered on a dielectric elastomer platforms actuated by electroactive nanofibers (FIG. 15). For example, dielectric elastomers known in the art, such as silicones (e.g., PDMS, rubbers) or acrylic elastomers (e.g., VHB 4910) can be fabricated as a thin sheet and two or more sheets can be stacked with a synthetic pigment structure and/or pigment granule to generate an elastomeric sac. The dielectric elastomer may be actuated with a suitable voltage field. On application of the voltage field, the synthetic chromatophore will compress in the transverse directions and expand to induce a color change. Multiple synthetic chromatophores can also be arranged in series to induce large scale color change.

Furthermore, as described in the appended examples, the synthetic pigment structures and isolated pigment granules described herein may be incorporated in nanofibers and nanofabrics using the devices and methods described in U.S. application Ser. No. 13/320,031 and PCT Application No. PCT/U.S. Ser. No. 11/061,241, the entire contents of each of which are incorporated herein by reference, as well as in 2-D and 3-D thin films as described herein and in, for example, U.S. Patent Publication no. 20090317852 U.S. application Ser. No. 13/318,227, the entire contents of each of which are also incorporated herein by reference.

This invention is further illustrated by the following examples which should not be construed as limiting. The contents of all references, patents and published patent applications cited throughout this application, as well as the Figures, are hereby incorporated herein in their entirety by reference.

### EXAMPLES

### Materials and Methods

The following materials and methods were used in the Examples below.

### Tissue Collection

Cuttlefish, S. officinalis, were collected and sacrified at the Marine Biological Laboratory in Woods Hole. Adult cuttlefish (1+ years old) were used. Tissue was removed from the dorsal mantle. The iridophore layer was separated from the chromatophore tissue, and the remaining chromatophore tissue was suspended in homogenization buffer. Chromatophore tissue was lysed by ultra-sonication and was purified in a sucrose gradient for up to 60 minutes. Final pellets were resuspended in homogenization buffer containing 10 mM Hepes buffer, 50 mM Potassium Aspartate, 10 mM magnesium chloride, and 1 mM dithiothreitol in PBS and stored at 4° C. until use.

### Protein Separation and Identification

Pigment pellets were resuspended in loading buffer containing 2% SDS in PBS, 50 mM Tris HCl, pH 6.8, 10% glycerol, 1% β-mercaptoethanol, 12.5 mM EDTA, and 0.02% bromophenol blue. Sodium dodecyl sulfate-polyacrylamide gel electrophoresis (SDS-PAGE) was used to separate protein fractions from the pellet. SDS PAGE was carried out on a Bio-Rad Mini-Protean II electrophoresis system (Bio-Rad, Hercules, Calif.).

The SDS-PAGE running buffer was composed of 28.8 g Glycine, 6.04 g Tris base, 2 g SDS, and 1.8 L water. The gels were 4-15% gradient polyacrylamide gels (Criterion Tris-HCl Gel Catalog #345-0028). The loading buffer contained 6% SDS. The gels were run at 100V for 10 minutes followed by 120V for 90 minutes.

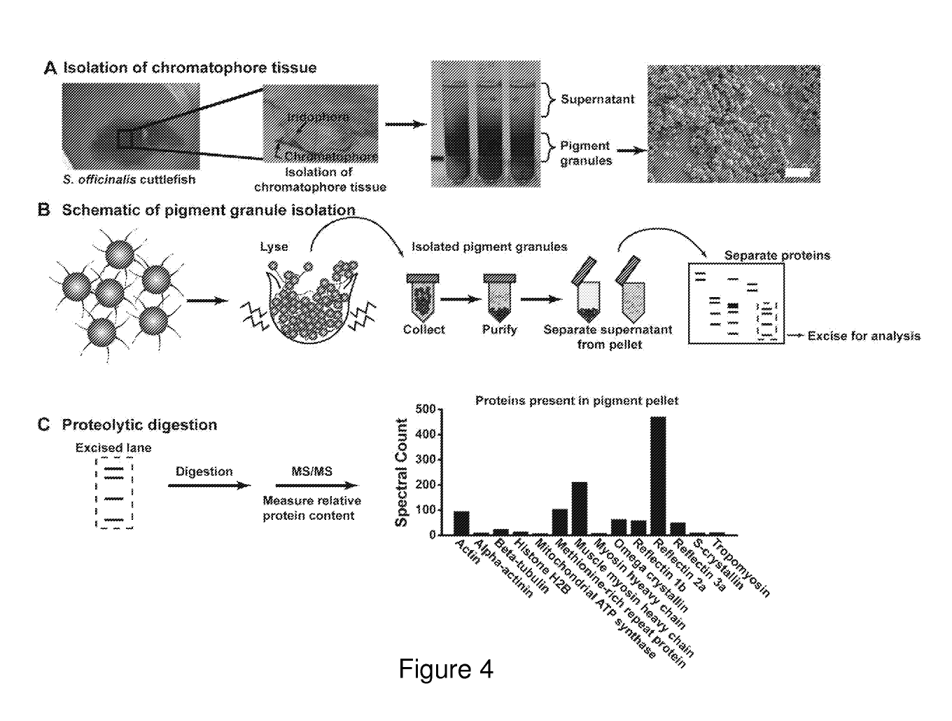

SDS-PAGE of the pigment pellets resulted in multiple protein bands ranging from about 0-75 kD. A whole lane containing proteins separated from the pigment pellet homogenate was excised from the gel and separated into 6 equivalent sections based on molecular weight. See FIG. 4D. Each section was washed in a 50:50 solution containing acetonitrile and water to remove any impurities and was then submitted for analysis.

Protein analysis was performed at the Harvard Mass Spectrometry and Proteomics Resource Laboratory, FAS Center for Systems Biology. Excised sections were subjected to tryptic digestion to remove the surrounding gel and cleave the purified peptides.

Cleaved peptides were analyzed by microcapillary reverse-phase HPLC, directly coupled to the nano-electrospray ionization source of an LTQ-Orbitrap Velos or LTQ-Oritrap XL mass spectrometer. These MS/MS spectra are correlated with known sequences using the algorithm Sequest. MS/MS peptide sequences were reviewed for homology to known proteins.

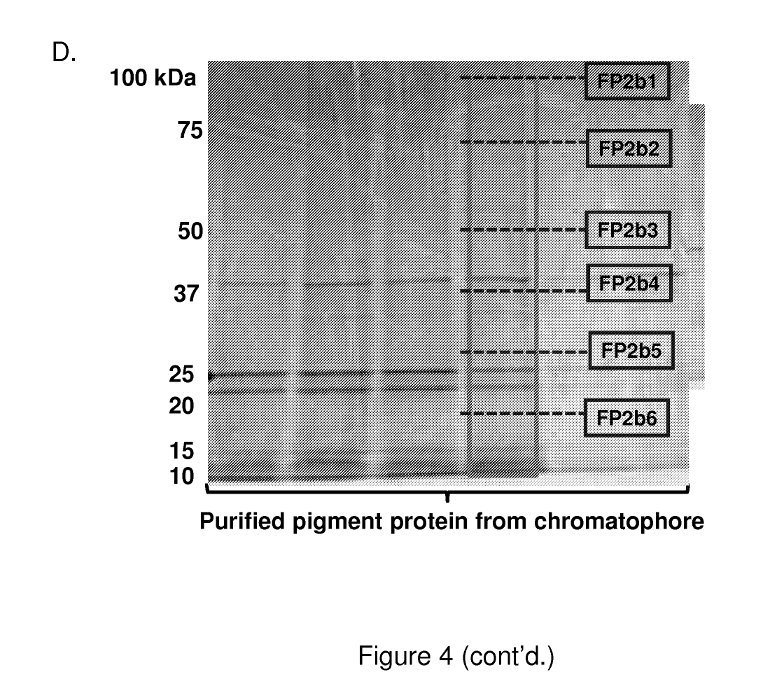

The relative protein content from the whole gel analysis revealed that pigment pellets are composed of reflectin isoforms, cytoskeletal proteins, microtubules, and crystallin isoforms (see FIG. 5C).

### Optical Measurements

Emission spectra were collected from isotropically aligned granules on 2-dimensional PDMS thin films using photoluminescence excitation (PLE) measurements. A confocal micro-Raman setup (LabAramis, Horiba) with 532 nm excitation was used for measurement of photoluminescence emission spectra. A custom built micro-photoluminescence setup was used for PLE measurements. Two different tunable lasers were used to span the excitation spectrum of the pigment granules. The doubled beam of a femtosecond-pulsed Ti:sapphire laser (MIRA, Coherent) was used for excitation wavelengths between 385 nm and 460 nm while a ps-pulsed supercontinum laser (SuperK, NKT Photonics) with Varia tunable filter was used for excitation wavelengths between 465 and 550 nm Samples were excited with a constant excitation power of approximately 80 μW. Emission spectra were collected in 5 nm excitation intervals between 385 nm and 550 nm; below 385 nm the objective was not transmissive. The peak emission was 720 nm.

Absorbance measurements were performed with a UV-Vis spectrometer (DU800 Beckman Coulter) in a 10 mm path length cuvette. Prior to measurement the pigment granule solution was agitated with a pipette.

### Simulations

Finite Difference Time Domain (FDTD) methods (FDTD Solutions, Lumerical) were used for computational studies of the importance of the nanostructuring of pigment granules on their optical properties.

### Example 1

### Sepia officinalis Pigment Granules and Pigment Proteins

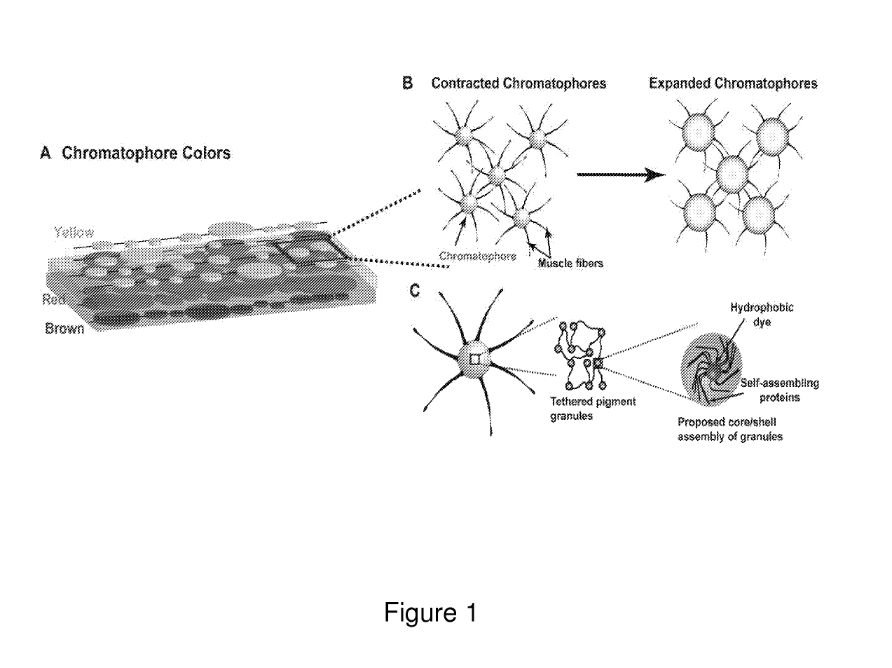

Nature has evolved unique mechanisms to rapidly change the color of an organism for communication and defense. For example, cuttlefish, Sepia officinalis, have evolved a highly effective mechanism of adaptive coloration with a millisecond response time (Mathger, L. M., et al. (2009) Journal of the Royal Society Interface 6, S149-S163; Mathger, L. M., Roberts, et al. (2010) Biology Letters 6, 600-603). This process relies on the cooperative assembly of three optical components within its dermal tissue (Mathger, L. M., et a. (2009) Journal of the Royal Society Interface 6, S149-S163; Cloney, R. A. & Brocco, S. L. (1983) Am. Zool. 23, 581-592 (1983); Sutherland, R. L., et al. (2008) Journal of the Optical Society of America-Optics Image Science and Vision 25, 2044-2054). The leucophore, a near-perfect scatterer, the iridophore, a Bragg stack reflector, and the chromatophore, a tunable color filter—all are involved in the rapid coloration of the cuttlefish (Sutherland, R. L., et al. (2008) Journal of the Optical Society of America-Optics Image Science and Vision 25, 2044-2054; Mathger, L. M. & Hanlon, R. T. (2007) Cell and Tissue Research 329, 179-186). While the leucophores and iridophores provide the adaptive base layer in the dermal tissue (Hanlon, R. T. & Messenger, J. B. (1998) Philosophical Transactions of the Royal Society of London Series B-Biological Sciences 320, 437), they are both passive components of coloration. The active coloration in S. officinalis is due to the chromatophore, which imparts color change through an aerial expansion of a pigment containing sacculus (Florey, E. (1969) Am. Zool. 9, 429; Florey, E. & Kriebel, M. E. (1969) Zeitschrift Fur Vergleichende Physiologic 65, 98). The spontaneous actuation of the chromatophore layer (FIG. 1A) in the dermis of cephalopods (cuttlefish, squid, octopus) is composed of an interpenetrating array of pigment granules localized within a cytoelastic sac (Mathger, L. M., et al. (2009) Journal of the Royal Society Interface 6, S149-S163; Mathger, L. M., Roberts, et al. (2010) Biology Letters 6, 600-603; Sutherland, R. L., et al. (2008) Journal of the Optical Society of America-Optics Image Science and Vision 25, 2044-2054; Mathger, L. M. & Hanlon, R. T. (2007) Cell and Tissue Research 329, 179-186; Florey, E. (1969)Am. Zool. 9, 429). The chromatophores undergo reversible actuation to induce both local and global patterning via a contraction and expansion mechanism along their length (FIG. 1B).

As such, these actuating chromatophores are a source of inspiration in designing adaptive coloration changes for the purposes of camouflage and communication. However, the major components that make up the chromatophore layer are still unknown (FIG. 1C). As described herein, the major proteins responsible for modulating and reflecting light within the dorsal mantle of the cuttlefish have been identified and a hierarchical mechanism of cuttlefish coloration focusing specifically on pigment granules as nanoscale photonic devices is described. In addition, the hierarchical mechanism of chromatophore coloration, as the nanoscale chromatophore pigment granules regulate absorbance, excitation, and scattering of light within the cuttlefish S. officinalis is described.

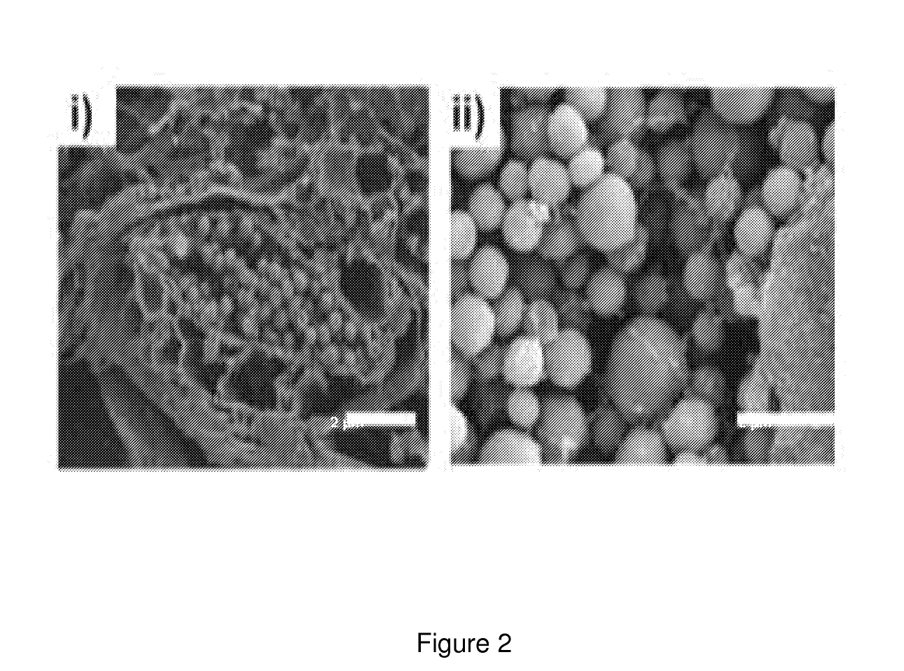

Until the discoveries described herein, pigment granules have been viewed as inert pigments localized within the chromatophore. For example, as depicted in FIG. 2, scanning electron microscopy shows that brown chromatophores from S. officinalis are composed of pigment granules that are nanospheres and have a diameter of about 930+/−250 nm. The pigment granules are tethered by microfilaments within the chromatophore cytoelastic sac and radial muscle fibers that anchor the cytoelastic sac are composed of actin.

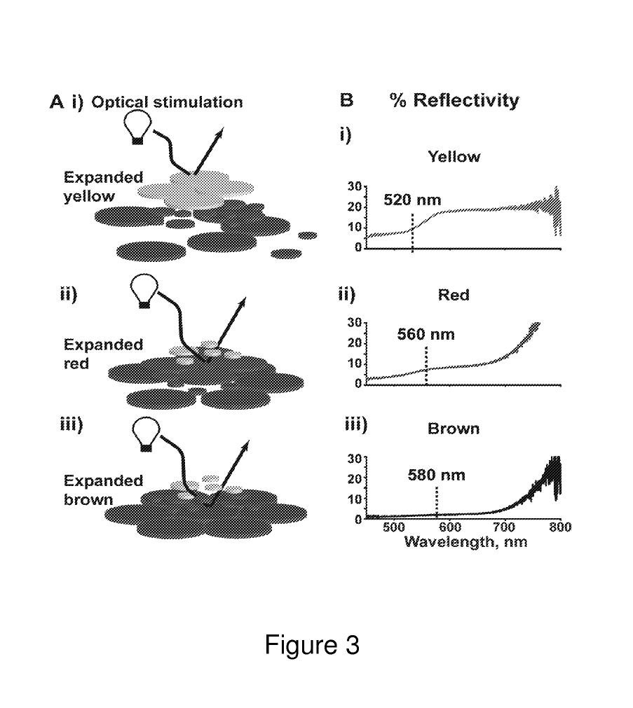

Due to the color dependent variation in size, shape and porosity of granules, it was hypothesized that the nanoscale pigment granules influence ultrafast coloration by selectively absorbing and scattering light (FIG. 3A). To test this hypothesis, the reflectivity of isolated S. officinalis chromatophores was measured. Maximum reflectivity from the yellow chromatophore was observed at 20%; whereas, a reflectivity of >5% was observed for the brown chromatophore (FIG. 3B). These data indicate that chromatophores are capable of scattering light, but the degree of scattered light depends on granule density dictated by chromatophore color.

The role that the pigment granules play in modulating the active coloration of chromatophores was also investigated. A procedure to isolate brown chromatophores and their associative pigment granules was, therefore, developed (FIG. 4A). Briefly, the epidermis and the iridophore layer were removed from a skin tissue sample and the remaining tissue, the chromatophore layer, was digested in collagenase. After digestion, the tissue was ultrasonicated (about 30 seconds) to homogenize it. The homogenate was then purified through a series of centrifugation and wash cycles (FIG. 5b). In particular, the homogenate was purified in a sucrose gradient (80%, 60%, and 20% sucrose). The tissue homogenate was placed on the sucrose gradient, spun at 150,000 g. Since brown chromatophores are densely packed with solid pigment granules, granules are easily separated from the red and yellow chromatophore homogenate, which remain in the supernatant. The supernatant was separated from the pigment pellet and stored for mass spectrometry analysis. Once the supernatant was removed, the pigment pellet was collected, sonicated for 20 minutes to break up the pellet, and further purified by 3 centrifugation and washing cycles. After the third rinse, the pigment pellet was resuspended in water. The chromatophores were lysed via ultrasonication, which is a process that is strong enough to rupture the chromatophore membrane but not strong enough to destroy the pigment granules.

Proteins associated with the pelleted pigment granules and supernatant were separated using gel electrophoresis, and the resultant protein bands were excised and analyzed using tandem Mass Spectrometry.

Proteins identified from the cuttlefish tissue had significant similarity (<50%) to the reflectin family of proteins (1a, 2a, 3) and crystallin proteins (FIG. 4C). Muscle proteins, such as actin and myosin, were also identified along with intermediate filament proteins (FIG. 4C).

The presence of novel isoforms of relectin proteins in S. officinalis skin was also observed within the lowest molecular weight (less than about 10 KD) band of the gel, indicating that novel optical proteins have been identified. MS/MS sequencing was used to identify these proteins specific to S. officinalis skin. Analysis of the sequences identified indicated that they have some amino acid sequence identity to known reflectins but represent new low molecular weight (<5 KD) reflectin isoforms (Table 1).

In order to investigate the optical properties of the pigment granules in the brown chromatophores of S. officinalis, 2-dimensional and 3-dimensional isotropic and anisotropic thin films containing the pigment granules isolated from the S. officinalis skin tissue were prepared. Thin films were prepared using inorganic substrates (e.g., silica, Au-coated silica, or sapphire substrates) and biopolymer substrates.

Methods to produce thin films are known in the art and described in detail in, for example, U.S. Publication No. 2009/0317852 and U.S. application Ser. No. 13/318,227, the entire contents of each of which are incorporated herein by reference.

For the biopolymer substrate, an inert polymer, polydimethylsiloxane (PDMS), was spin-coated onto glass in a uniform layer (about 5 μm to about 25 μm thick). Once the polymer layer was cured, it was stamped using a PDMS stamp having 10 μm wide lines spaced 10 μm apart with a mixture of proteins or peptides, such as fibronectin, laminin, poly-L-lysine and/or collagen, and the pigment granules isolated from the brown chromnatophores of S. officinalis tissue to in order produce anisotropic 2-D patterns.

For isotropic 2-D patterning, the pigment granules isolated from the brown chromatophores of S. officinalis tissue were evenly distributed onto PDMS-coated coverslips by incubating a dilute solution of about 0.5 mg/mL of pigment granules on the coverslips for about 45 minutes. The average size of one pigment granule immobilized on the PDMS-coated substrate was determined to be about 820 nm+/−230 nm, which is on the order of a wavelength of visible light indicating that these pigment granules have the ability to scatter light (the graph in FIG. 5).

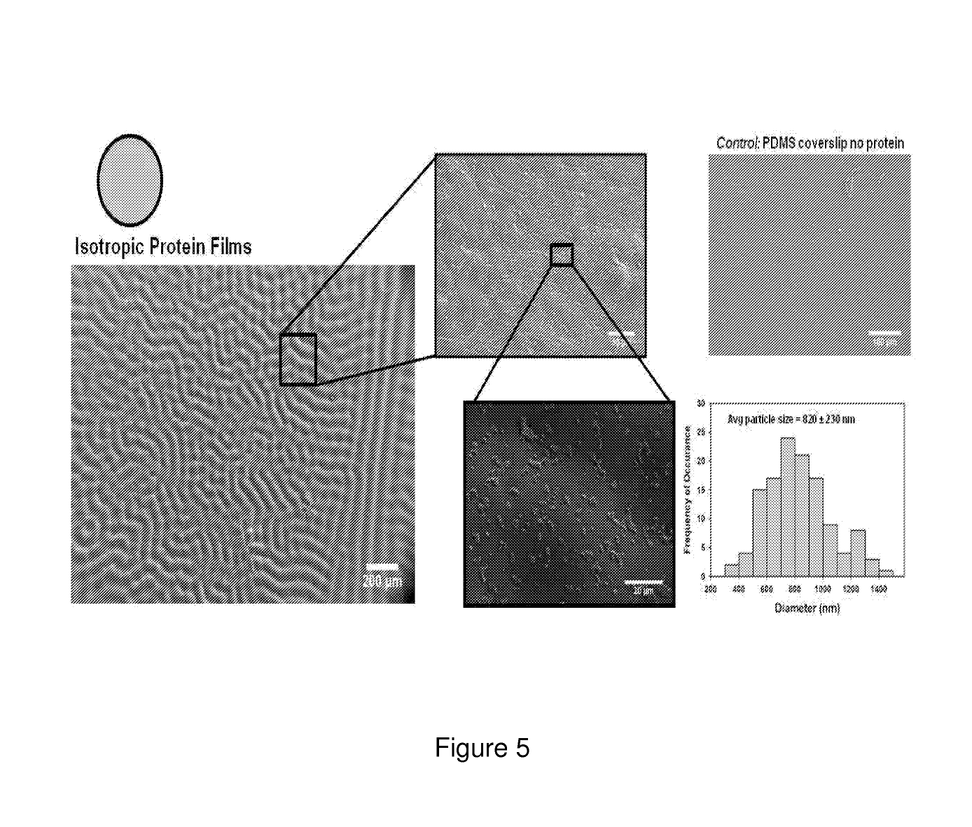

In order to determine if the pigment granules assemble in a similar manner when embedded in a 3-D matrix, pigment granules isolated from the brown chromatophores of S. officinalis skin were also embedded within a micropatterned 3-D matrix comprising alginate. Granules were first encapsulated within the aqueous based alginate gel. The loaded gel was then cross-linked using a modified version of microcontact printing to simultaneously stamp and crosslink the alginate with a calcium chloride loaded agar stamp (See FIG. 6).

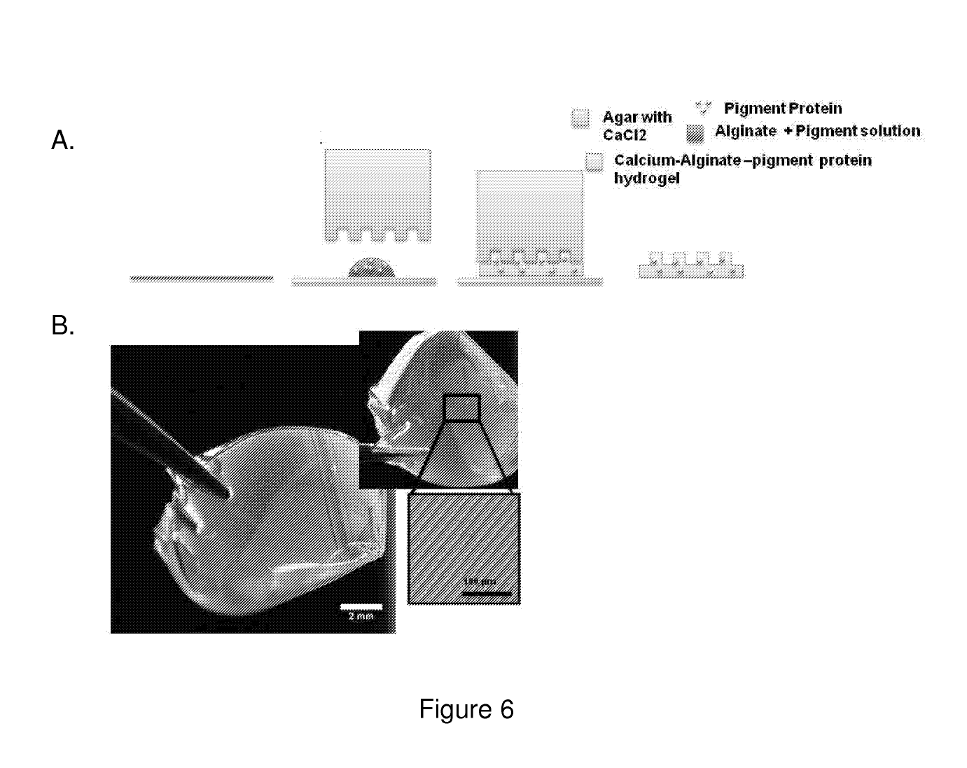

The optical properties of the pigment proteins in the 2-D thin films (pigment granules immobilized on PDMS) and the 3-D alginate gels was measured using Micro-Photoluminescence (MicroPL) which permits the optical behavior of a single pigment granule to be observed. MicroPL is a technique that excites a sample with a femto-second light pulse (400-450 nm) and emission (530-800) spectra is collected. MicroPL demonstrated that the pigment granules not only absorb light but also reemit light at about 650 nm to about 700 nm when excited with light at about 410 or about 532 nm. Furthermore, MicroPL indicates the pigment granules isolated from the brown chromatophores luminesce, a property never before observed for cephalopod chromatophores (FIG. 7).

A surface initiated fiber assembly process or the Rotary Jet Spinning (RJS) processes to form optically active nanoFabrics and nanofibers was also used to investigate the optical properties of the pigment granules isolated from the brown chromatophores of S. officinalis skin. These techniques enable the formation of biological or bio-composite fibers with a dynamic size range (e.g., about 10 nm to about 200 nm thickness) and are described in detail in U.S. application Ser. No. 13/320,031 and PCT Application No. PCT/U.S. Ser. No. 11/061,241, the entire contents of each of which are incorporated herein by reference. Briefly, the process includes spin-coating a temperature sensitive polymer, such as poly(N-isopropylacrylamide) (PIPAAm) onto a glass coverslip in a uniform layer, adsorbing nanometer-thick layers of soluble proteins, such as fibronectin, onto a hydrophobic surface at high density to partially unfold them and expose cryptic binding domains, thermally triggering surface dissolution to synchronize matrix assembly and non-destructive release, and transferring fibers to be used as nanoscale optical textiles. As the temperature of the reaction solution is lowered below 35° C., PIPAAm dissolves, and the fibers release into solution. At this step the substrate can be agitated to assist in release of fibers, and a standard syringe can be used to place them onto elastomeric membranes or other flexible substrates.

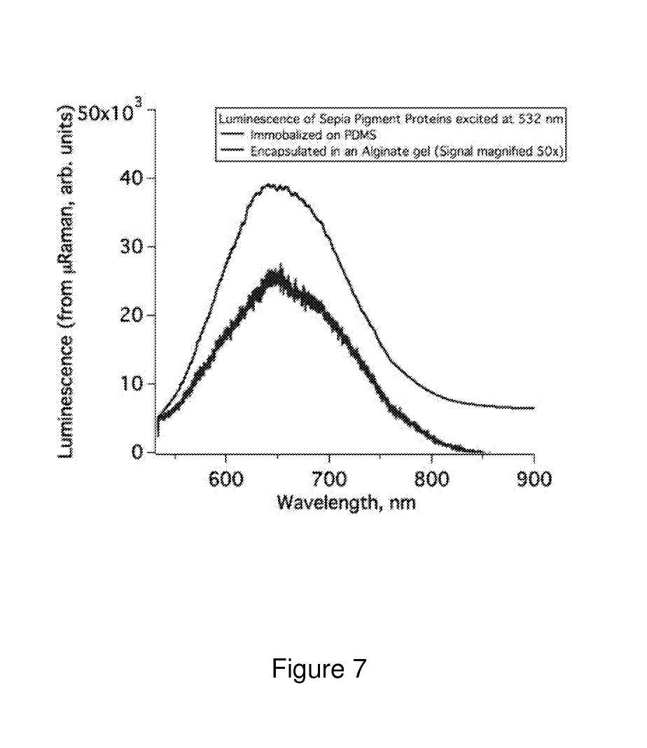

For the preparation of nanofibers, 20 mg/mL of the pigment granules isolated from the brown chromatophores suspended in 7.2 wt % poly-lactic acid (PLA) rotated at about 30,000 rpm using a device comprising rotational motion, such as the device depicted in FIG. 8.

MicroPL of the fibers indicated that the emission profile had a peak centered at about 700 nm.

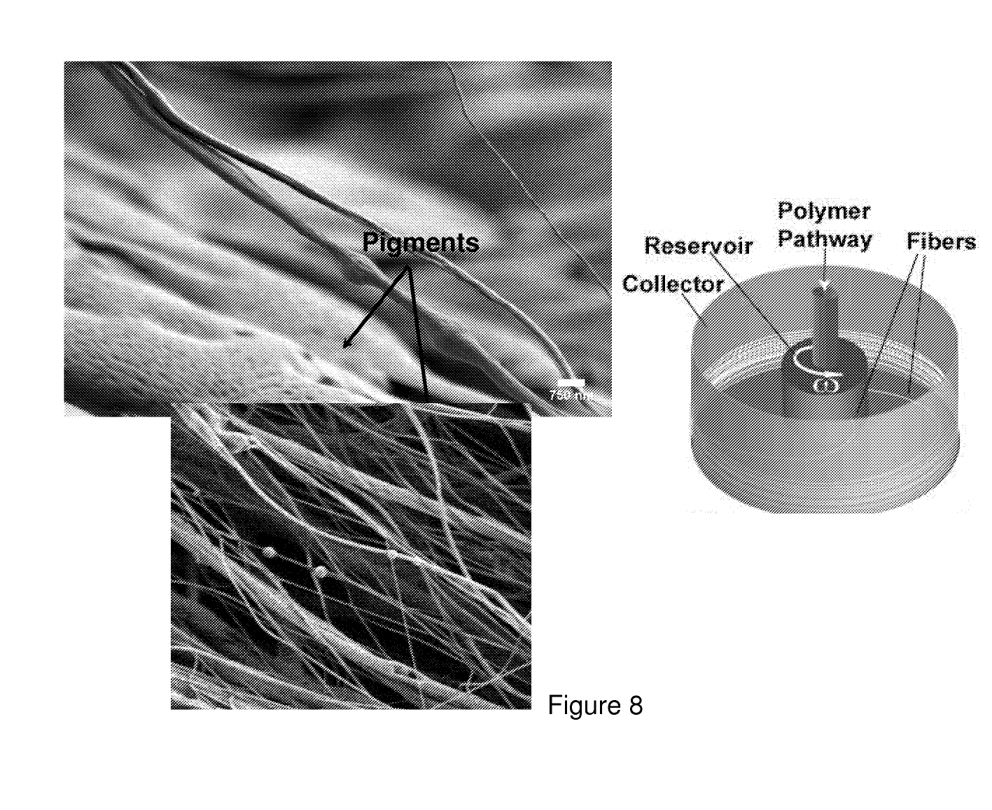

The excitation and emission spectra of the isotropically aligned pigment granules on a PDMS substrate, the pigment granules embedded in alginate gels, and in textiles were determined by exciting the pigment granules with the blue/green light (about 410-532 nm), and collected the emission spectra in far red (600-900 nm). FIG. 9 shows that the excitation sweep (dotted line) was performed using the doubled beam of a femtosecond-pulsed Ti:Sapphire laser. Samples were excited through a 0.95 NA, 100× objective with a constant excitation power of approximately 80 μW. Emission spectra were collected in 5 nm excitation intervals between 385 nm and 460 nm; below 385 nm the objective was not transmissive and above 460 nm the stability of the laser decreased. The light gray line is the standard deviation. Peak emission is 700 nm (dark gray line).

The luminescence of the pigment granules on PDMS was probed via microphotoluminescence spectroscopy (μ-PL). Using μ-PL enabled the excitation of and collection from single pigment granules. Fluorescence measurements were performed on 2D isotropically distributed brown pigment granules on PDMS.

A confocal micro-Raman setup (LabAramis, Horiba) with 532 nm excitation was used for measurement of PL emission spectra. A custom built micro-photoluminescence setup was used for the photoluminescence excitation (PLE) measurements. Two different tunable lasers were used to span the excitation spectrum of the pigment granules.

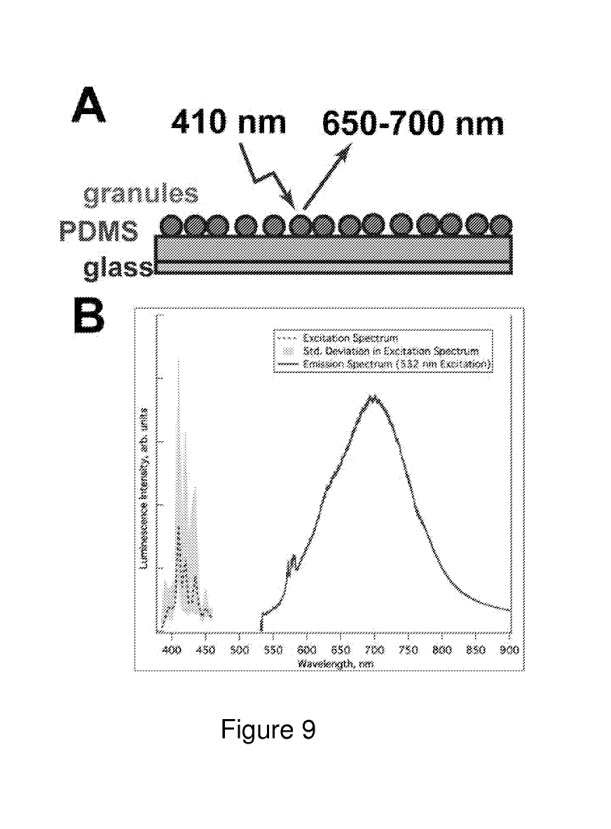

The data demonstrate that the photoluminescence spectra of the pigment granules shifts to a higher wavelength depending on the material that the pigment granules are embedded in or on (FIG. 10).

The analysis of the optical properties of the pigment granules processed under variable reaction conditions demonstrates that there is a unique far-red fluorescence associated with the pigment granules. Fluorescence, which requires an external light source which excites the material stimulating the emission of lower energy light, is different than a bioluminescence, which is often observed by bacteria producing substances via a chemical reaction or oxidation. Other biomolecules that fluoresce include GFP and its analogues, oxidized melanins, or the lipofuscin. Most reported bioluminescence is blue, and is shifted to higher wavelength through the secondary excitation of another fluorophore (GFP for example). Recently it was discovered that a deep sea fish, the dragonfish does exhibit red luminescence stimulated by blue bioluminescence. This luminescence is the result of the excitation of a red pigment that is related to bacteriochlorophyll.

The observed luminescence of the pigment granules isolated from the brown chromatophores has a very large Stokes shift. The Stokes shift is a measure of the difference in wavelength between excitation and emission. This is quite exotic for fluorescence in a biological system, and only recently has a red-emitting, blue-excited fluorescent protein been discovered, red fluorescent protein or RFP which is man-made molecule not one isolated from a biological organism.

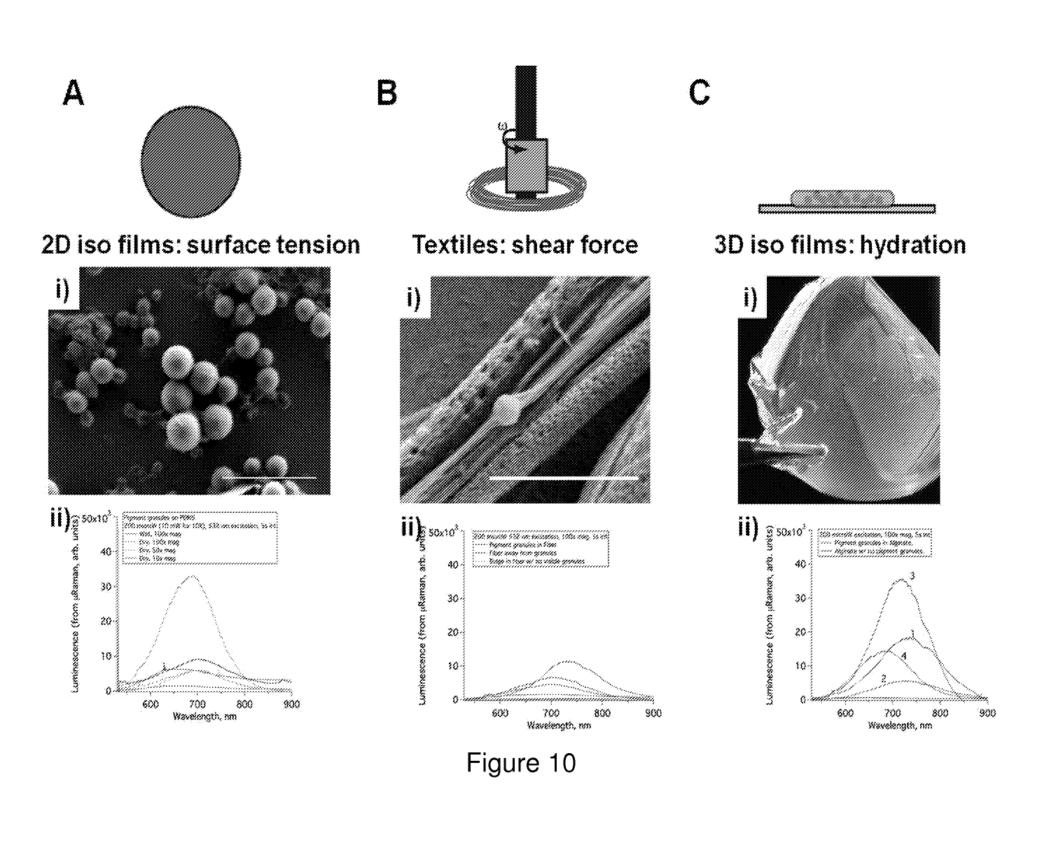

In order to determine how the chemical composition and granular architecture of the pigment granules isolated from the brown chromatophore of S. officinalis skin influence cuttlefish coloration, granules were denatured in varying concentrations of sodium hydroxide (NaOH), which hydrolyzes amino acid esters or amides. Both the visible color (FIG. 11A i) and absorbance (FIG. 11A ii) of granules are altered in the presence of NaOH. SEM was used to follow the change in granular ultrastructure as a function of increasing NaOH concentration (FIG. 11B). It was observed that addition of 0.1M NaOH enhances surface roughness of granules. At 0.2M NaOH, the spherical geometry of granules is destroyed. At 0.5-0.8M NaOH, the structure of granules appears to restructure, in a manner reminiscent of an inverse micelle. Thus, visible coloration, absorbance, and scattering are all dependent on granular architecture of the pigment granule. Gel electrophoresis of denatured granules also indicates that there is a loss of protein content (FIG. 11C).

In order to determine which proteins are associated with the optical changes of the pigment granules, proteins associated with the denatured granules were separated using gel electrophoresis, and global mass spectral analysis was used to identify proteins lost during denaturation. A maximum protein signal at 0.2M NaOH denaturation was observed, indicating that proteins have been released from granules. Reflectin isoforms 2a and 3a, methionine-rich repeat proteins, and cobalt-nitrile hydratase are all reduced as granular structure is broken down in increasing NaOH. These data indicate that granular structure plays a greater role than protein composition in absorbing and scattering light.

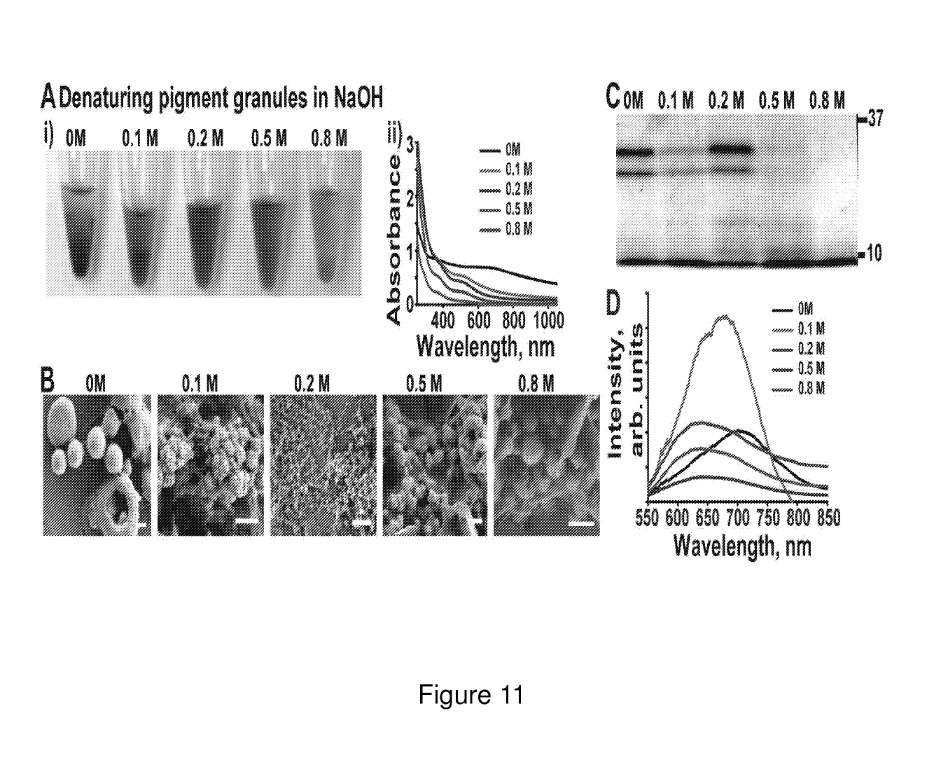

To determine if the nanospherical structure of pigment granules protects reflective proteins and enhances luminescence, luminescence was measured for the denatured granules. Upon interaction with NaOH, the emission maximum blue-shifted, centering broadly between 600-675 nm (FIG. 11D). The maximum intensity was observed at 0.1M NaOH treatment, likely due to the increased surface area exposed on the granules. Emission intensity minimum was observed at 0.2M. This is due to the complete disruption of the granular structure at 0.2M NaOH (FIG. 12).

Collectively, these data demonstrate that the majority of the ability of the chromatophore pigment granule to modulate light is the granular structure of the pigment granule and that protein composition of the pigment granule plays a lesser role.

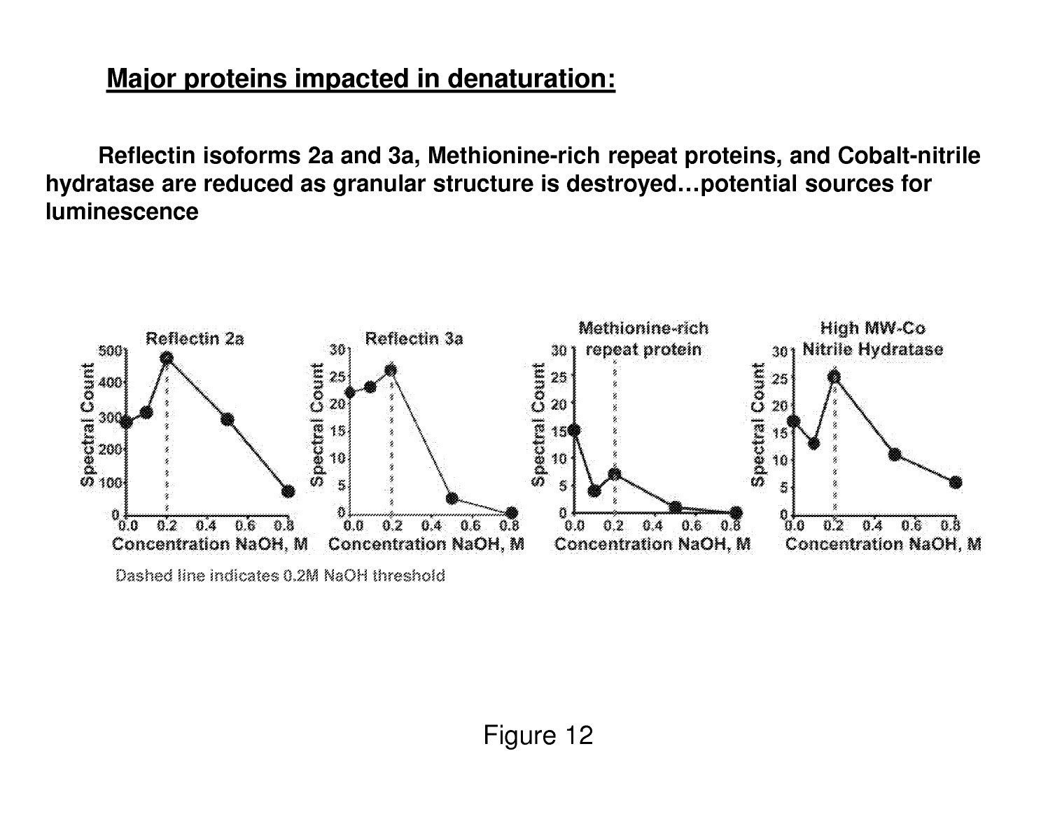

In order to demonstrate how the high refractive index, reflectin-based pigment granules enhances coloration and reflectance, a finite difference time domain model was built. The modeled “chromatophore” was composed of 5 micron thick layer of absorbing spheres, a radius of about 250 to about 700 nm, and absdorbace from the UV and visible spectrum. The model demonstrated that the higher refractive index increase reflectivity and color contrast mimicking reflectivity of granules in chromatophore organs. The model also demonstrated that the random arrangement and distribution of the spheres results in angular independence no interference effects. The model, thus, supports the experimental reflectivity data and indicates that high refractive index material (e.g., reflectin proteins) must be present within the pigment granules (FIG. 13).

Thus, the isotropic arrangement and broad size distribution of pigment granules in chromatophores of S. officinallis leads to large optical contrast through the combination of light scattering and absorbance. The nanoscale geometry of the pigment granules means that light experiences a longer path length as it travels through the chromatophore structure, thereby enhancing absorbance by the pigment contained within the granule. The absorbance by the pigment eliminates angular effects and minimizes spectral variation with thickness of the granular layer.

In summary, cuttlefish camouflage is dependent on the molecular level assembly of reflective proteins and pigments over multiple spatial scales. The composition and nanospherical granular geometry of pigments within the chromatophore provide the increase reflectivity and color contrast in granules required for the ultra-fast changes in coloration. Reflectin is one protein associated with pigment granules. Pigment arrangement may be random within chromatophore but granular architecture is highly evolved to produce maximum reflectivity and coloration in cuttlefish. Nanospherical structure of pigment granule protects reflective proteins and enhances luminescence.

### EQUIVALENTS

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

### INCORPORATION BY REFERENCE

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

What is claimed is:

1. A pigment structure, comprising a reflectin protein and a light absorbing material.

2. The pigment structure of claim 1, wherein the light absorbing material is a dye and/or flurorophore.

3. The pigment structure of claim 2, wherein the dye is an inorganic dye.

4. The pigment structure of claim 2, wherein the dye is an organic dye.

5. The pigment structure of claim 1, wherein the reflectin protein is selected from the group consisting of Euprymna scolopes reflectin 1a, Euprymna scolopes reflectin 1b, Euprymna scolopes reflectin 2a, Euprymna scolopes reflectin 2b, Euprymna scolopes reflectin 2c, Euprymna scolopes reflectin 3a, Doryteuthis pealeii reflectin-like A1, Doryteuthis pealeii reflectin-like A2, and Doryteuthis pealeii reflectin-like B1.

6. A method for preparing a pigment structure, comprising
providing a reflectin protein and a material having a lower reflective index than the reflectin protein,
combining the reflectin protein and the material having a lower reflective index than the reflectin protein under conditions such that a reflectin protein nanosphere comprising the material having a lower reflective index than the reflectin protein forms, thereby preparing a pigment structure.

7. The method of claim 6, wherein the material having a lower reflective index than the reflectin protein is a dye.

8. The method of claim 7, wherein the dye is an inorganic dye.

9. The method of claim 7, wherein the dye is an organic dye.

10. The method of claim 6, wherein the reflectin protein is selected from the group consisting of Euprymna scolopes reflectin 1a, Euprymna scolopes reflectin 1b, Euprymna scolopes reflectin 2a, Euprymna scolopes reflectin 2b, Euprymna scolopes reflectin 2c, Euprymna scolopes reflectin 3a, Doryteuthis pealeii reflectin-like A1, Doryteuthis pealeii reflectin-like A2, and Doryteuthis pealeii reflectin-like B1.

11. An isolated pigment granule, wherein the pigment granule is isolated from a brown chromatophore of the chromatophore skin layer of Sepia officianalis.

12. The isolated pigment granule of claim 11, wherein the pigment granule has a diameter of about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, or about 2000 nm.

13. The isolated pigment granule of claim 11, which fluoresces.

14. The isolated pigment granule of claim 11, which when excited with light having a wavelength of about 400, 401, 402, 403, 404, 405, 406, 407, 408, 409, 410, 411, 412, 413, 414, 415, 416, 417, 418, 419, or about 420 nm has a maximum emission at about 620, 630, 640, 650, 660, 670, 680, 690, 700, 710, 720, 730, 740, or about 750 nm.

15. The isolated pigment granule of claim 11, which when excited with light having a wavelength of about 400, 401, 402, 403, 404, 405, 406, 407, 408, 409, 410, 411, 412, 413, 414, 415, 416, 417, 418, 419, or about 420 nm has a maximum emission between about 620-750, 620-720, 650-750, 620-700, or about 650-700 nm.

16. The isolated pigment granule of claim 11, which when excited with light having a wavelength of about 522, 523, 524, 525, 526, 527, 528, 529, 530, 531, 532, 533, 534, 535, 536, 537, 538, 539, 540, 541, or about 542 nm has a maximum emission at about 620, 630, 640, 650, 660, 670, 680, 690, 700, 710, 720, 730, 740, or about 750 nm.

17. The isolated pigment granule of claim 11, which when excited with light having a wavelength of about 522, 523, 524, 525, 526, 527, 528, 529, 530, 531, 532, 533, 534, 535, 536, 537, 538, 539, 540, 541, or about 542 nm has a maximum emission between about 620-750, 620-720, 650-750, 620-700, or about 650-700 nm.

18. An isolated Sepia officinalis pigment protein, comprising the amino acid sequence YQDMMNMDFHGR (SEQ ID NO:1).

19. An isolated Sepia officinalis pigment protein, comprising the amino acid sequence YDNYGHDQYHGR (SEQ ID NO:2).

20. An isolated Sepia officinalis pigment protein, comprising the amino acid sequence LMYNNMYR (SEQ ID NO:3).

21. The isolated protein of any one of claims 18-20, having a molecular weight less than about 10 kD.

22. The isolated protein of claim 21, having a molecular weight less than about 5 kD.

23. The isolated protein of claim 21, having a molecular weight less than about 3 kD.

24. An isotropic thin film comprising the pigment structure of claim 1.

25. A nanofiber comprising the pigment structure of claim 1.

26. A textile comprising the pigment structure of claim 1.

27. A sensor comprising the pigment structure of claim 1.

28. A colorant comprising the pigment structure of claim 1.

29. A therapeutic comprising the pigment structure of claim 1.

30. A cosmetic comprising the pigment structure of claim 1.

31. A food product comprising the pigment structure of claim 1.

32. A device for diagnostic and/or therapeutic purposes comprising the pigment protein of claim 1.

33. An isotropic thin film comprising the isolated pigment granule of claim 11.

34. A nanofiber comprising the isolated pigment granule of claim 11.

35. A textile comprising the isolated pigment granule of claim 11.

36. A sensor comprising the isolated pigment granule of claim 11.

37. A colorant comprising the isolated pigment granule of claim 11.

38. A therapeutic comprising the isolated pigment granule of claim 11.

39. A cosmetic comprising the isolated pigment granule of claim 11.

40. A food product comprising the isolated pigment granule of claim 11.

41. A device for diagnostic and/or therapeutic purposes comprising the isolated pigment granule of claim 11.


SEQUENCE LISTING (3 sequences)

SEQ ID NO: 1 - protein, 12 aa, Artificial Sequence
  Sepia Officinalis Partial Pigment Protein Sequence
YQDMMNMDFHGR

SEQ ID NO: 2 - protein, 12 aa, Artificial Sequence
  Sepia Officinalis Partial Pigment Protein Sequence
YDNYGHDQYHGR

SEQ ID NO: 3 - protein, 8 aa, Artificial Sequence
  Sepia Officinalis Partial Pigment Protein Sequence
LMYNNMYR